(19) United States
(12) Patent Application Publication
(10) Pub. No.: US 2011/0212954 A1
(43) Pub. Date: Sep. 1, 2011

(54) ALPHA-LIPOIC ACID DERIVATIVES AND THEIR USE IN DRUG PREPARATION

(76) Inventors: Mario Brufani, Castel Gandolfo (IT); Ilaria Medici, Roma (IT); Rinaldo Marini Bettolo, Roma (IT); Luisa Maria Migneco, Roma (IT); Rolando Marzella, Roma (IT); Rocco Figliola, Montefalcone Di Valforte (IT); Angela La Bella, Roma (IT)

(21) Appl. No.: 13/128,193

(22) PCT Filed: Nov. 6, 2009
(86) PCT No.: PCT/EP09/64770

§ 371 Date: May 6, 2011

(30) Foreign Application Priority Data
Nov. 7, 2008 (EP) 08425716.1

(51) Int. Cl. A61K 31/5377; C07D 339/04; A61K 31/385; C07D 409/06; A61K 31/4535; A61K 31/496; C07D 413/06; A61P 3/04; A61P 3/10; A61P 35/00

(52) U.S. Cl. 514/231.5; 549/39; 514/440; 546/207; 514/326; 544/374; 514/252.13; 544/145

(57) ABSTRACT

The present invention concerns an enantiomer R of a compound of Formula (I), wherein X is —NH—R₁ or of Formula (V) or (VI), R₁ is —(CH₂)ₙ—R₂, R₂ is a linear, branched or cyclic C₁C₆ aliphatic group, —O—(CH₂)ₙ—CH₃, —NH—CO—(CH₂)ₙ—CH₃, a 5- or 6-membered aliphatic or aromatic ring optionally comprising a heteroatom, a 5- or 6-membered aromatic ring substituted by one or two substituents, said substituents being selected from the group consisting of —OH, —O(alkyl C₁C₃) and —OCO(alkyl C₁C₃), or of Formula (V), R₃ is H or a C₁-C₃ aliphatic group and R₄ is a linear C₁-C₃ or a branched C₃-C₁₂ aliphatic group, or R₃ is a C₁-C₃ aliphatic group and R₄ is a linear C₁-C₁₂ aliphatic group, Y is O, CH—(CH₂)ₙ—CH₃ or N(CO)(CH₂)ₙ—CH₃, and n is an integer from O to 6. It has been found that the enantiomers of the invention are able to release (R)-alpha-lipoic acid, ensuring a longer permanence in the body for the pharmacologically active principle than that obtainable by its direct administration, or to simulate the pharmacological action of alpha-lipoic acid itself, while exhibiting a much more intense and lasting activity.

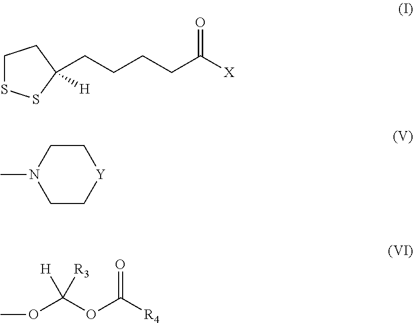

### FIELD OF THE INVENTION

The present invention concerns new derivatives of alpha-lipoic acid (α-LA) having improved pharmaceutical properties and a higher bioavailability than alpha-lipoic acid as such. In particular, said derivatives find use in the treatment of diabetes, diabetic neuropathy and obesity.

### STATE OF THE ART

Alpha-lipoic acid is a cofactor for several oxidative decarboxylation reactions of alpha-keto acids such as pyruvic acid, alpha-ketoglutaric acid, branched-chain alpha-keto acids and glycine.

Alpha-lipoic acid (α-LA or 1,2-dithiolane-3-pentanoic acid, or 1,2-dithiolane-3-valeric acid or thioctic acid) (formula A) in its R enantiomeric form is bound to the oxidative decarboxylase multienzyme complexes of alpha-keto acids (alpha-keto acid dehydrogenase), where it carries out oxidation-reduction functions by enzymatically reducing to alpha-dihydrolipoic acid (α-DHLA) (formula B):

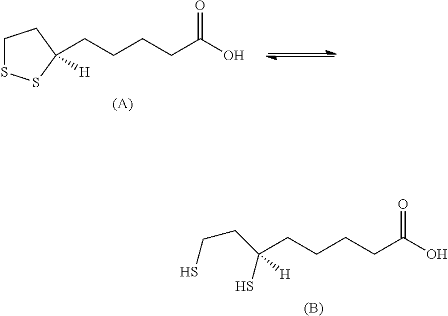

and reoxidizing, enzymatically again, to α-LA (formula A).

Alpha-lipoic acid also acts as a transporter of acetyl residues; in fact, it transfers the acetyl group, which forms by oxidative decarboxylation of pyruvic acid, to Coenzyme A. The reaction, which requires α-LA as cofactor, can be schematically represented as shown below:

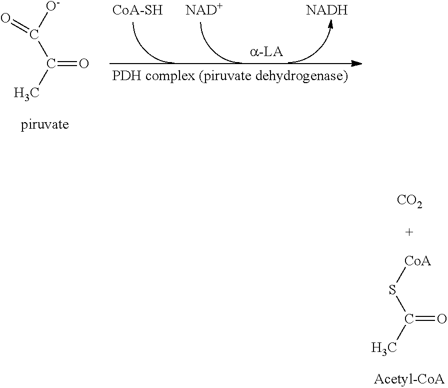

This reaction scheme shows that the oxidizing agent is NAD⁺ and that the reaction produces one equivalent of NADH. The reaction takes place in the mitochondria and is essential for starting Krebs Cycle reactions.

α-LA, particularly in its racemic form, is widely used as a food supplement, and in some Countries as a drug for treating diabetic polyneuropathy. The basis for the pharmacological action of alpha-lipoic acid is still unclear, although in this sense several hypotheses have been advanced. In particular, alpha-lipoic acid has been hypothesized to have a protective effect in neuropathic processes due to its oxidation-reduction properties capable, at least partly, to neutralize the damage caused by free radicals generated in the peripheral nervous system of the diabetic patient as a consequence of glucose reduction to sorbitol and the latter reoxidation to fructose. Recently, it has been shown in pharmacological models of diabetes type II and by using high doses of α-LA (30 mg/kg in rats), that alpha-lipoic acid also has a direct anti-diabetic action; in fact, it reduces glycemia in diabetic rats, increases entry of glucose into its muscle cells and suppresses glucose synthesis in hepatic cells.

The feasibility of using α-LA as a drug is, however, limited by its unfavourable pharmacokinetic characteristics. In this respect, α-LA exhibits in man a plasma half life (t_{1/2}) of 28 minutes, as well as a bioavailability, after oral administration, of less than 30%. The easiness with which alpha-lipoic acid is metabolized by oxidative means (primarily by beta-oxidation) is probably responsible for these unfavourable pharmacokinetic characteristics. The R enantiomer of α-LA is less toxic and pharmacologically more active than the corresponding raceme, but nevertheless exhibits the same unfavourable pharmacokinetic characteristics as racemic α-LA. The object of the present invention is, therefore, to provide alpha-lipoic acid in a way that it persists in the body in a greater amount than that obtainable by its direct administration, with improved bioavailability, and at the same time exhibits a more intense and lasting activity.

### SUMMARY OF THE INVENTION

Such an object has been achieved by an enantiomer R of a compound of formula I:

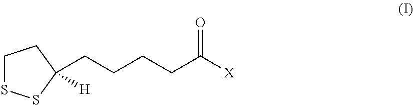

wherein

X is —NH—R₁ or

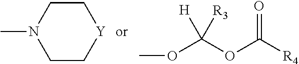

R₁ is —(CH₂)ₙ—R₂,

R₂ is a linear, branched or cyclic C₁-C₆ aliphatic group, —O—(CH₂)ₙ—CH₃, —NH—CO—(CH₂)ₙ—CH₃, a 5- or 6-membered aliphatic or aromatic ring optionally comprising a heteroatom, a 5- or 6-membered aromatic ring substituted by one or two substituents, said substituents being selected from the group consisting of —OH, —O(alkyl C₁-C₃) and —OCO(alkyl C₁-C₃), or

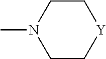

R₃ is H or a C₁-C₃ aliphatic group and R₄ is a linear C₁-C₃ or a branched C₃-C₁₂ aliphatic group, or R₃ is a C₁-C₃ aliphatic group and R₄ is a linear C₁-C₁₂ aliphatic group,

Y is O, CH—(CH₂)ₙ—CH₃ or N(CO)(CH₂)ₙ—CH₃, and

n is an integer from 0 to 6.

Said enantiomers are able to release alpha-lipoic acid, thus ensuring a greater permanence in the body of the pharmacologically active principle than that obtainable by its direct administration, or to simulate the same pharmacological action of alpha-lipoic acid itself, but exhibiting a much more intense and lasting activity.

In a further aspect, the present invention concerns the use of said enantiomers in the treatment of diabetes, diabetic neuropathy, obesity and pathologies related thereto.

In an even further aspect, the present invention concerns the use of said enantiomers for inducing apoptosis of tumour cells in the treatment of tumours.

### BRIEF DESCRIPTION OF THE FIGURES

The characteristics and advantages of the present invention will be evident from the detailed description given below, from the embodiments provided by way of non-limiting illustration, and from the accompanying figures, wherein:

FIG. 1 shows cell viability expressed in %, measured after treatment with the compounds of the present invention at concentrations of 500 μM;

FIG. 2 shows the result of in vitro enzymatic hydrolysis assays of some of the enantiomers R of the present invention after 1 h and 3 h;

FIG. 3 shows the result of in vitro enzymatic hydrolysis assays of some of the enantiomers R of the present invention after 24 h;

FIG. 4 shows cell viability expressed as “fold induction” relative to that induced by (R)alpha-lipoic acid, measured after treatment with compounds of the present invention at concentrations of 1 mM;

FIG. 5 shows the results obtained in vivo relating to the amount of plasmatic alpha-lipoic acid after the treatment of some compounds of the present invention;

FIG. 6 shows the amount of some of the compounds measured in vivo over time;

FIG. 7 shows the results obtained in vivo relating to the amount of plasmatic alpha-lipoic acid after the treatment of different compounds of the present invention;

FIG. 8 shows the amount of some of the compounds measured in vivo over time;

FIG. 9 shows the results obtained in vivo relating to the amount of plasmatic alpha-lipoic acid after the treatment with two enantiomers of the invention;

FIG. 10 shows the amount of NADH, expressed as “fold induction”, compared to the control (DMSO), measured with varying concentrations of some of the compounds of the present invention; and

FIG. 11 gives the amount of NADPH, expressed as “fold induction”, compared to treatment with control DMSO, measured with varying concentrations of some of the compounds of this invention.

### DETAILED DESCRIPTION OF THE INVENTION

The invention, therefore, relates to (R)-α-LA derivatives able to release (R)-α-LA or to simulate its pharmacological action. In particular, the invention concerns an enantiomer R of a compound of formula I:

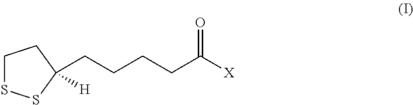

wherein

X is —NH—R₁ or

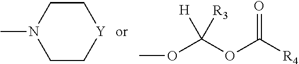

R₁ is —(CH₂)ₙ—R₂,

R₂ is a linear, branched or cyclic C₁-C₆ aliphatic group, —O—(CH₂)ₙ—CH₃, —NH—CO—(CH₂)ₙ—CH₃, a 5- or 6-membered aliphatic or aromatic ring optionally comprising a heteroatom, a 5- or 6-membered aromatic ring substituted by one or two substituents, said substituents being selected from the group consisting of —OH, —O(alkyl C₁-C₃) and —OCO(alkyl C₁-C₃), or

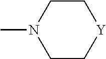

R₃ is H or a C₁-C₃ aliphatic group and R₄ is a linear C₁-C₃ or a branched C₃-C₁₂ aliphatic group, or R₃ is a C₁-C₃ aliphatic group and R₄ is a linear C₁-C₁₂ aliphatic group,

Y is O, CH—(CH₂)ₙ—CH₃ or N(CO)(CH₂)ₙ—CH₃, and

n is an integer from 0 to 6.

It has been surprisingly found that the enantiomer R of compounds of formula I overcome the problems deriving from the rapid metabolization of alpha-lipoic acid, as they are able to release alpha-lipoic acid itself to hence ensure a longer permanence of the pharmacologically active principle than that obtainable by its direct administration, or to simulate its pharmacological action while exhibiting a more intense and lasting activity, as will become more evident from the examples given below. Furthermore, the compounds of formula I have the enantiomeric form R, since it has been surprisingly found that even for these derivatives this enantiomeric form is significantly less toxic and pharmacologically advantageously more active than the corresponding racemic form.

In a preferred embodiment, the enantiomer R of the compounds has formula III:

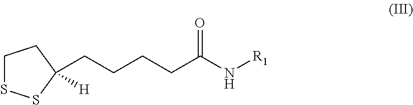

wherein

R₁ is —(CH₂)ₙ—R₂,

R₂ is a linear, branched or cyclic C₁-C₄ aliphatic group, and n is 0.

The enantiomer R of these compounds surprisingly exhibit very high plasma level even over 3 hours from the administration, as well as a bioavailability significantly higher than the bioavailability shown by the alpha-lipoic acid as such, as will become evident from the Examples given below.

The enantiomers R according to said preferred embodiment have been subjected to enzymatic hydrolysis tests able to demonstrate α-LA release both in vitro and in vivo (Examples 30 and 31, respectively).

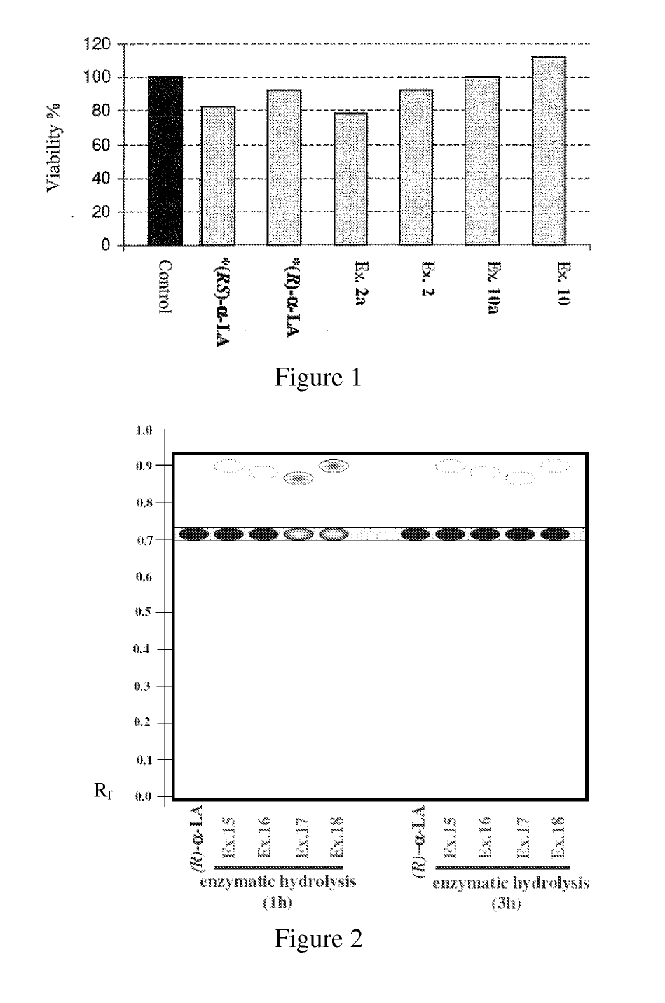

As it appears from said Examples, with specific regard to FIGS. 2-3 and 5-9, the bioavailability and the t_{1/2} of α-LA released both in vitro and in vivo from the enantiomers R of the invention are significantly greater than those obtainable with (R)-α-LA directly administered. Said enantiomers can hence find advantageous application as prodrugs of α-LA since they are able to release it in vivo and to significantly increase its bioavailability and its permanence in the body.

According to said preferred embodiment, the preferred enantiomers R have formula:

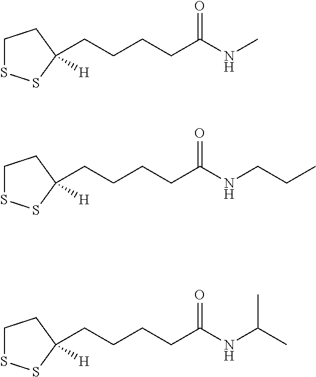

The more preferred enantiomers R have formula:

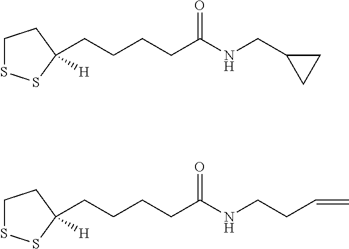

The most preferred enantiomer R has formula:

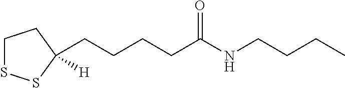

As a matter of fact, in the Examples 29-31 with reference to FIGS. 1, 3, 4, and 7-9, this enantiomer shows the best combination of results in terms of amount of released (R)-α-lipoic acid, period of release, bioavailability and cell viability. In another preferred embodiment, the enantiomer R of the compounds has formula III:

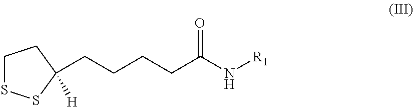

wherein

R₁ is —(CH₂)ₙ—R₂,
R₂ is —NH—CO—(CH₂)ₙ—CH₃, a 5- or 6-membered aliphatic ring, a 5-membered aromatic ring, a 5- or 6-membered aromatic ring substituted by one or two substituents, said substituents being selected from the group consisting of: —OH, —O(alkyl C₁-C₃) and —OCO(alkyl C₁-C₃), or

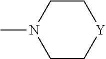

where Y is CH—(CH₂)ₙ—CH₃ or N(CO)(CH₂)ₙ—CH₃, and
n is an integer from 0 to 6, or
R₂ is phenyl and n is an integer from 2 to 6, or
R₂ is morpholinyl and n is an integer from 3 to 6, or
R₂ is —O—(CH₂)ₙ—CH₃ and n is an integer from 1 to 6,

or

R₁ is a linear, branched or cyclic C₅-C₁₀ aliphatic group.

The enantiomers R according to said another preferred embodiment have been also subjected to enzymatic hydrolysis tests able to demonstrate α-LA release both in vitro and in vivo (Examples 30 and 31, respectively).

According to said another preferred embodiment, the preferred enantiomers R have formula:

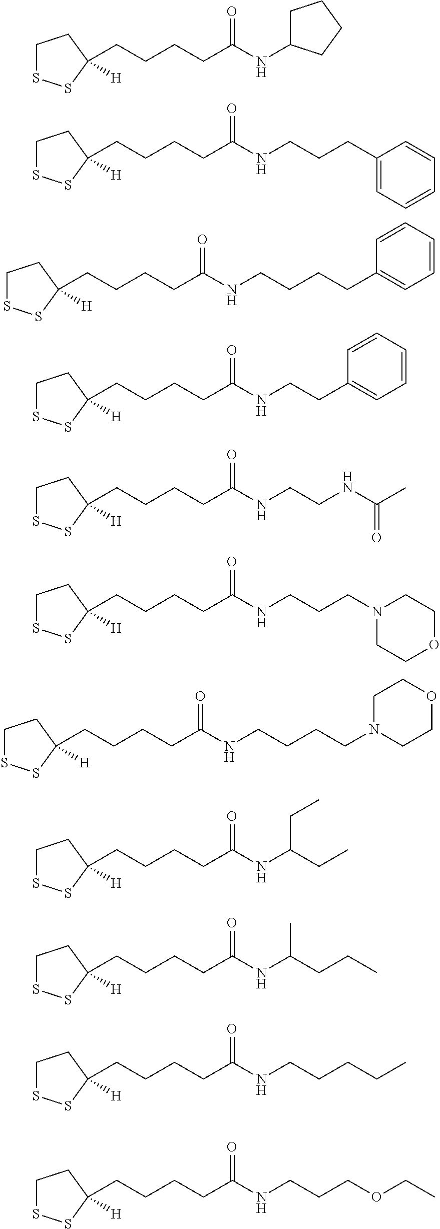

The most preferred enantiomers R have formula:

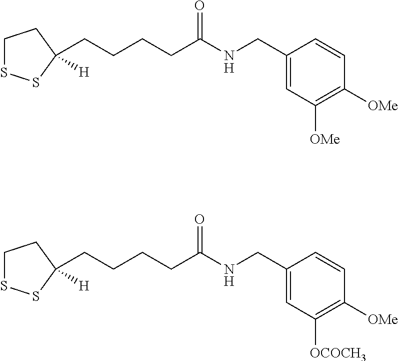

More preferably, the enantiomers R of said another preferred embodiment have formula III, wherein R₁ is a linear, branched or cyclic C₇-C₁₀ aliphatic group. Particularly preferred is the enantiomer R having formula:

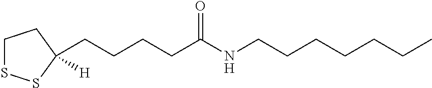

In a further preferred embodiment, the enantiomer R of the compounds has formula II:

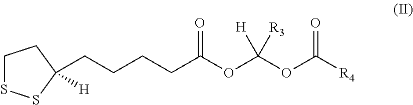

wherein R₃ is H or a C₁-C₃ aliphatic group and R₄ is a branched C₃-C₁₂ aliphatic group, wherein at least a branch is in alpha-position, or

R₃ is a C₁-C₃ aliphatic group and R₄ is a linear C₁-C₆ aliphatic group.

The enantiomer R of these compounds surprisingly exhibit very high plasma level even over 3 hours from the administration, as well as a bioavailability significantly higher than the bioavailability shown by the alpha-lipoic acid as such, as will become evident from the Examples given below. Therefore, these enantiomers R have proved to be advantageously suitable, differently from alpha-lipoic acid as such, for controlled release formulations.

According to said further preferred embodiment, the preferred enantiomers R have formula:

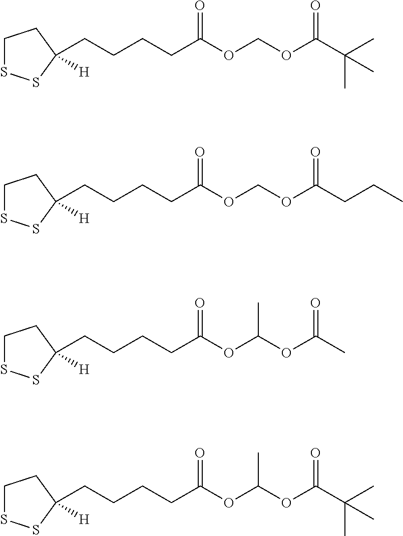

In a further preferred embodiment, the enantiomer R of the compounds has formula IV:

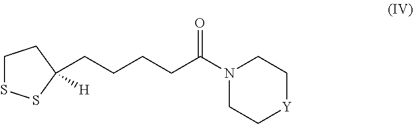

wherein

Y is —CH—(CH₂)ₙ—CH₃ or —N(CO)(CH₂)ₙ—CH₃, and n is an integer from 0 to 3.

According to said still another preferred embodiment, the preferred enantiomers R have formula:

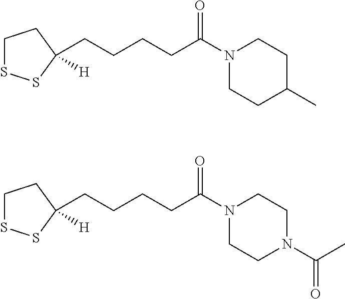

In an another aspect, the present invention concerns a process for preparing the enantiomer R of the compound of formula I, comprising the step of reacting (R)-alpha-lipoic acid and a reagent under inert gas atmosphere and room temperature, sheltered from light, wherein said reagent is selected from the group consisting of NH₂—R₁,

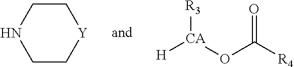

wherein

R₁ is —(CH₂)ₙ—R₂,

R₂ is a linear, branched or cyclic C₁-C₆ aliphatic group, —O—(CH₂)ₙ—CH₃, —NH—CO—(CH₂)ₙ—CH₃, a 5- or 6-membered aliphatic or aromatic ring optionally comprising a heteroatom, a 5- or 6-membered aromatic ring substituted by one or two substituents, said substituents being selected from the group consisting of —OH, —O(alkyl C₁-C₃) and —OCO(alkyl C₁-C₃), or

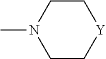

R₃ is H or a C₁-C₃ aliphatic group and R₄ is a linear C₁-C₃ or a branched C₃-C₁₂ aliphatic group, or R₃ is a C₁-C₃ aliphatic group and R₄ is a linear C₁-C₁₂ aliphatic group,

Y is O, CH—(CH₂)ₙ—CH₃ or N(CO)(CH₂)ₙ—CH₃,

A is a halogen, and

n is an integer from 0 to 6.

Preferably, said (R)-alpha-lipoic acid and reagent are reacted in equimolar amounts.

In a further aspect, the present invention concerns the enantiomer R of a compound of formula I:

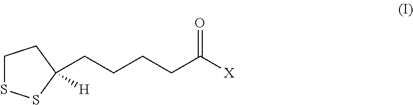

wherein

X is —NH—R₁ or

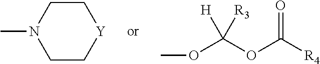

R₁ is —(CH₂)ₙ—R₂,
R₂ is a linear, branched or cyclic C₁-C₆ aliphatic group, —O—(CH₂)ₙ—CH₃, —NH—CO—(CH₂)ₙ—CH₃, a 5- or 6-membered aliphatic or aromatic ring optionally comprising a heteroatom, a 5- or 6-membered aromatic ring substituted by one or two substituents, said substituents being selected from the group consisting of —OH, —O(alkyl C₁-C₃) and —OCO(alkyl C₁-C₃), or

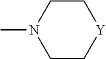

R₃ is H or a C₁-C₃ aliphatic group and R₄ is a linear C₁-C₃ or a branched C₃-C₁₂ aliphatic group, or R₃ is a C₁-C₃ aliphatic group and R₄ is a linear C₁-C₁₂ aliphatic group,

Y is O, CH—(CH₂)ₙ—CH₃ or N(CO)(CH₂)ₙ—CH₃, and

n is an integer from 0 to 6,

for use as a medicament.

Specifically, the present invention also concerns the use the enantiomer R of the compound of formula I:

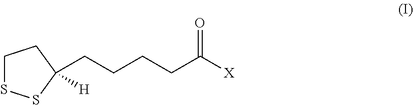

wherein

X is —NH—R₁ or

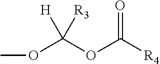

R₁ is —(CH₂)ₙ—R₂,
R₂ is a linear C₁-C₃ aliphatic group and n is an integer from 0 to 2, or
R₂ is a branched or cyclic C₁-C₆ aliphatic group, —O—(CH₂)ₙ—CH₃, —NH—CO—(CH₂)ₙ—CH₃ and n is an integer from 0 to 6, or
R₂ is a 5- or 6-membered aliphatic or aromatic ring optionally comprising a heteroatom, or a 5- or 6-membered aromatic ring substituted by one or two substituents, said substituents being selected from the group consisting of —OH, —O(alkyl C₁-C₃) and —OCO(alkyl C₁-C₃), or

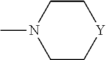

n is an integer from 0 to 6, and
Y is O, CH—(CH₂)ₙ—CH₃ or N(CO)(CH₂)ₙ—CH₃,

R₃ is H or a C₁-C₃ aliphatic group and R₄ is a branched C₃-C₁₂ aliphatic group, wherein at least a branch is in alpha-position, or

R₃ is a C₁-C₃ aliphatic group and R₄ is a linear C₁-C₆ aliphatic group, for the production of a medicament for the treatment of diabetes, diabetic neuropathy, obesity and pathologies related thereto.

The enantiomers R as above defined are secondary amides, among which the preferred enantiomers R have formula:

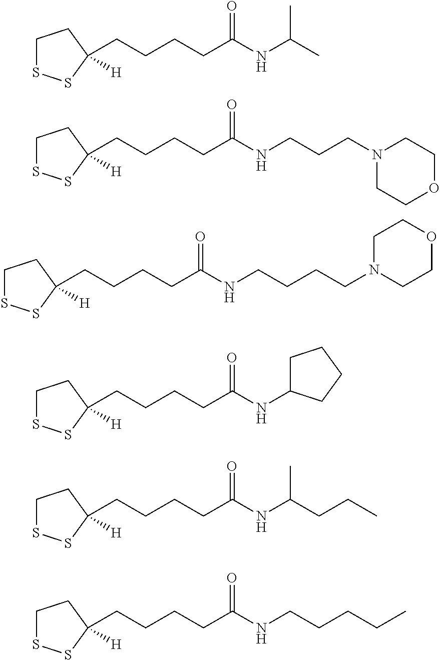

It has been surprisingly found that the enantiomer R of compounds of formula I as above defined, besides being significantly less toxic and pharmacologically advantageously more active than the corresponding racemic forms, overcome the problems deriving from the rapid metabolization of alpha-lipoic acid, as they are able to release alpha-lipoic acid itself to hence ensure a longer permanence of the pharmacologically active principle than that obtainable by its direct administration, or to simulate its pharmacological action while exhibiting a more intense and lasting activity. In this regard, in this first case, the enantiomers R can be successfully used as pro-drugs, whereas in the second case, when the enantiomers R are not hydrolysable or are hydrolysable excessively slowly, they can find advantageous application as (R)-α-LA analogous drugs, as they have been shown to have significantly more intense and lasting pharmacological activity than (R)-α-LA as such in the treatment of type II diabetes, diabetic neuropathy and obesity.

Particularly preferred for the above indicated use is the enantiomer R of the compound of formula I:

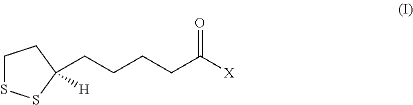

wherein

X is —NH—R₁ or

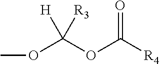

R₁ is —(CH₂)ₙ—R₂,
R₂ is a linear, branched or cyclic C₁-C₃ aliphatic group, and n is 1, or
R₂ is —NH—CO—(CH₂)ₙ—CH₃, —O—(CH₂)ₙ—CH₃, a 5- or 6-membered aliphatic or aromatic ring, a 5- or 6-membered aromatic ring substituted by one or two substituents, said substituents being selected from the group consisting of: —OH, —O(alkyl C₁-C₃) and —OCO(alkyl C₁-C₃), or

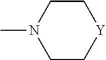

where Y is O, CH—(CH₂)ₙ—CH₃ or N(CO)(CH₂)ₙ—CH₃, and
n is an integer from 1 to 6,

R₃ is H or a C₁-C₃ aliphatic group and R₄ is a branched C₃-C₁₂ aliphatic group, wherein at least a branch is in alpha-position,

or

R₃ is a C₁-C₃ aliphatic group and R₄ is a linear C₁-C₆ aliphatic group.

Specifically, when R₁ is —(CH₂)ₙ—R₂, the enantiomers R of the invention are secondary amides, wherein at least one methylenic group is present at the alpha position to the amide nitrogen, as “n” always denotes at least 1. The inventors have surprisingly found that lipoamidase, i.e. the enzyme that in nature hydrolyses the bond between α-LA and the NH₂ residue of the lysine of the E2 enzyme in the pyruvate dehydrogenase multienzyme complex, hydrolyse these specific secondary amides, so that (R)-alpha-lipoic acid is advantageously slowly released. Actually, these enantiomers R surprisingly exhibit a detectable plasma level even over 3 hours from the administration, as well as a bioavailability significantly higher than the bioavailability shown by the (R)-alpha-lipoic acid as such, as will become evident from the Examples given below. Said enantiomers R can hence find advantageous application as prodrugs of (R)-α-LA since they are able to release it in vivo and to significantly increase its bioavailability and its permanence in the body.

With regard to treatment of diabetes, diabetic neuropathy, and pathologies related thereto, it is known that (RS)-α-LA and also (R)-α-LA, when administered to diabetes-induced rats, lowers plasma glucose levels. Some experiments have shown that (RS)-α-LA activates AMPK, the key energy homeostasis enzyme in the body. This enzyme is activated when cellular AMP levels are elevated and those of ATP are low, i.e. when the cell is in an energy deficit state. As AMPK sensitises muscle and hepatic cells to insulin action, it has been hypothesized that the anti-diabetic action of α-LA can at least be partly ascribable to its AMPK activation capability.

Without wishing to be bound by any theory, the inventors of the present invention hypothesize that AMPK activation by α-LA is the consequence of NADH depletion which α-LA induces in muscle and hepatic cells. In this respect, NADH supplies the energy for ATP synthesis. According to tests carried out by the inventors, the decrease in NADH levels would take place under a dual mechanism. Firstly, α-LA utilizes NADH as a reducing agent, to undergo reduction to α-DHLA. Secondly, exogenous α-LA, through the mass effect, at high concentrations blocks the oxidative decarboxylation reaction of pyruvate and leads to the reduction of NAD⁺ to NADH according to the following reaction:

α-DHLA+NAD⁺⇄α-LA+NADH

The presence of high concentrations of exogenous α-LA reverses this reaction which is the last one in the process of reactions leading to the oxidative decarboxylation of pyruvate. It has already been experimentally proven on various cell cultures that exogenous α-LA at low concentrations accelerates and at high concentrations slows down the overall oxidative decarboxylation process of pyruvic acid. The effect of exogenous α-LA has also been studied, on various cell models, on each of three enzymes which constitute the pyruvate dehydrogenase complex.

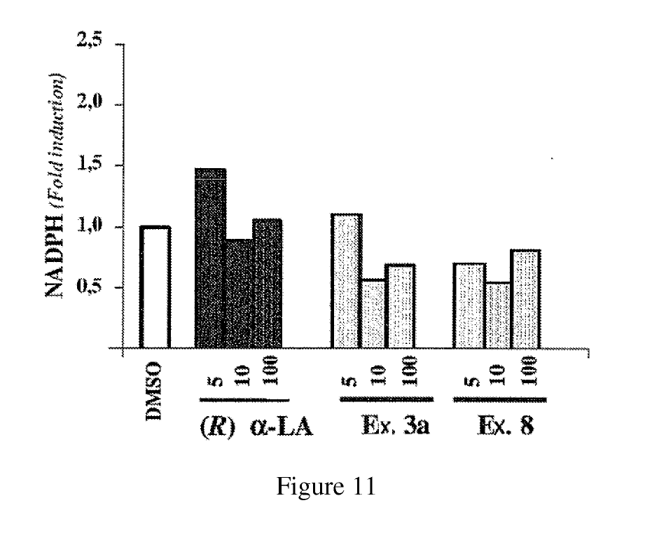

As can be seen in Example 32 below, using human hepatic cells (HepG2), suitable tests have been devised for demonstrating the effects of (R)-α-LA and the enantiomers of the present invention on cellular NADH levels. As also shown in FIGS. 10 and 11, the enantiomers R of the present invention, either non hydrolysable or weakly hydrolysable, result in the same effects on NADH as does (R)-α-LA but at significantly lower concentrations than (R)-α-LA, hence conveniently with a higher safety margin than (R)-α-LA as such.

Studies carried out on pharmacological models of diabetic neuropathy and controlled clinical studies have shown alpha-lipoic acid to have a protective action, ascribed to its antioxidant capability. At the same time, the use of synthetic antioxidants with the same oxidation-reduction activities, but with structures different from alpha-lipoic acid, has not given the expected results. Excess glucose present in the peripheral nervous system of the diabetic patient gives rise to non-enzymatic glycosylation of nerve cell proteins, thus altering their functional characteristics. Moreover, excess glucose is partly reduced to sorbitol by the aldose reductase enzyme which uses NADPH as reducing agent. Sorbitol is then oxidized to fructose, using NAD⁺ as oxidizing agent. The free radicals produced during the two oxidation-reduction reactions above described are among the main causes of neuropathic damage. Based on these considerations, the inventors of the present invention, supported by various data in the literature, hypothesize that in cells sensitive to diabetic damage (those of the nervous system, retina, kidneys), hyperglycemia induces depletion of NADPH and accumulation of NADH. The result is a reduction in efficiency of the antioxidant systems based on the balance of oxidized glutathione/reduced glutathione and dependent on the availability of NADPH.

In this situation, it has been hypothesized that α-LA and its reduction product α-DHLA are able to neutralize the typical oxidative damage of diabetic neuropathy by rebalancing the NADPH/NADH ratio. In this respect, α-LA is reduced to α-DHLA by means of lipoamide dehydrogenase which uses only NADH as reducing agent. It has been shown that α-DHLA directly reduces oxidized glutathione and other oxidation products, thus contributing to the restoration of NADPH to physiological levels.

For a description of the methods for analysing levels of NADH and NADPH, reference is made to Example 32 wherein the ability of (R)-α-LA and the enantiomers R of the present invention to influence NADH and NADPH levels are compared.

With regard to obesity, the inhibiting effect of α-LA on hunger mechanisms has already been demonstrated. In this respect, α-LA has been shown to inactivate the AMPK enzyme in appetite-controlling hypothalamic cells and the mechanisms by which AMPK stimulates appetite. As the limited effect of α-LA, and similarly of (R)-α-LA, on appetite mechanisms is attributed on its brief plasma half-life and its low bioavailability, the enantiomers R of formula I of the present invention, preferably the secondary amides, also find advantageous application in the treatment of obesity.

In a further aspect, the present invention concerns the use of the enantiomer R of the compound of formula I:

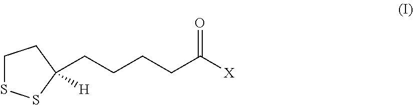

wherein

X is —NH—R₁ or

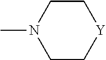

R₁ is a linear C₆-C₁₂ aliphatic group, or is a branched C₅-C₁₂ aliphatic group, wherein at least an ethyl branch is in alpha-position,

Y is O, CH—(CH₂)ₙ—CH₃ or N(CO)(CH₂)ₙ—CH₃, and

n is an integer from 0 to 6,

for the production of a medicament for inducing apoptosis of tumour cells in the treatment of tumours.

The enantiomers R encompassed by the above definition are secondary amides having long linear or specifically branched aliphatic chains or tertiary amides. Among these enantiomers R, the following are preferred:

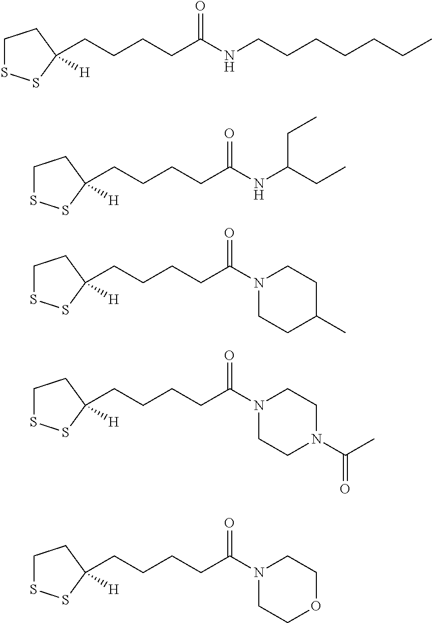

In this respect, the inventors have surprisingly found that lipoamidase, i.e. the enzyme that in nature hydrolyses the bond between α-LA and the NH₂ residue of the lysine of the E2 enzyme in the pyruvate dehydrogenase multienzyme complex, does not hydrolyse these amides which remain essentially unaltered in the body for a long time.

The long permanence of said amides in the body also indicates that these latter, are less susceptible to β-oxidation than α-LA. It should be reminded that the brief plasma half-life of α-LA in the body is mainly due to its oxidation at the β position; its main metabolite, found in tests on rats after oral administration, is actually 3-keto lipoic acid which is recovered in the blood at higher concentrations than α-LA. The above amide enantiomers R of this embodiment have been found to be cytotoxic in tests of cell viability, as reported in the following Example 29. This finding can be ascribable to the oxidative stress that these enantiomers can raise in cells. It should be noted that the alpha-lipoic acid itself has been found to cause oxidative stress in cells when in high concentrations, that results in apoptosis induction in several kind of tumour cells (Simbula et al., “Increased ROS generation and p53 activation in a-lipoic acid-induced apoptosis of hepatoma cells”, Apoptosis 2007, 12: 113-123, and Choi et al. “Mechanism of α-lipoic acid-induced apoptosis of lungs cancer cells”, Ann. N.Y. Acad. Sci. 2009, 1171: 149-155), but not in the non transformed cells, as NIH 3T3 fibroblasts.

Indeed, the enantiomers R as above defined have been found to induce an oxidative stress and accordingly to show a cytotoxic action in tumour cells greatly higher than the α-lipoic acid as such.

The following Examples of the present invention are provided by way of non-limiting illustration.

### EXAMPLES

### Preparation of the Compounds of Examples 1-14 and 19-28

A two-neck round-bottom flask equipped with magnetic stirrer was flame-heated under argon flow then covered with silver paper to avoid exposure to light. A solution of (RS)-alpha-lipoic acid (Examples 2a, 3a, 10a) or (R)-alpha-lipoic acid (Examples 1-14 and 19-28) (1 mole) in DMF was then prepared. The solution was stirred and the amine (1 mole) followed by the EDAC (1.1 mole) were then added. The resulting mixture was maintained at room temperature under argon atmosphere with stirring for about 2 hours.

The reaction mixture was then transferred into a separating funnel, having taken care to previously cover the glassware to be used with silver paper to avoid exposing the solution to light. After washing with saline, the aqueous phase was extracted with Et₂O (4×10 ml) and the pooled organic phases were dried over anhydrous Na₂SO₄, filtered and evaporated under reduced pressure. The mixture thus obtained was then transferred to a dark coloured flask and maintained under a high vacuum pump (24 hours) to remove DMF. The mixture was then purified over a chromatography column (SiO₂, CHCl₃: 100%). The alpha-lipoic acid derivative was then separated and characterized by GC-MS analysis and ¹H-NMR, ¹³C-NMR and IR spectroscopy.

### Example 1

### (R)-1-morpholin-4-yl-5-[1,2]dithiolan-3-yl-pentan-1-one

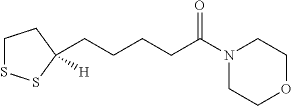

Yield: 40%

¹H-NMR (CDCl₃) 300 MHz: 3.66-3.36 (m, 9H), 3.19-3.00 (m, 2H), 2.48-2.35 (m, 1H), 2.27 (t, J=7.3, 2H), 1.94-1.79 (m, 1H), 1.73-1.34 (m, 6H); ¹³C-NMR (CDCl₃) 75 MHz: 171.2, 66.7, 66.4, 56.2, 45.8, 41.7, 40.0, 38.3, 34.5, 32.6, 28.9, 24.7; IR (CCl₄) ν_{C═O} 1657 cm⁻¹; GC-MS: 275 m/z (M^{+•}).

### Example 2

### (R)-5-[1,2]dithiolan-3-yl-pentanoic acid butylamide

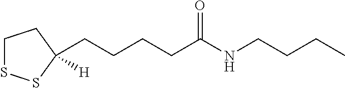

Yield: 80%

¹H-NMR (CDCl₃) 300 MHz: 5.70 (s, 1H), 3.57-3.48 (m, 1H), 3.23-3.05 (m, 4H), 2.47-2.37 (m, 1H), 2.16-1.77 (m, 3H), 1.70-1.20 (m, 10H), 0.88 (t, J=7.20, 3H); ¹³C-NMR (CDCl₃) 75 MHz: 172.6, 56.3, 40.1, 39.1, 38.4, 36.4, 34.5, 31.6, 28.8, 25.4, 20.0, 13.7; IR (CCl₄) ν_{N═H} 3465 cm⁻¹, ν_{C═O} 1662 cm⁻¹; GC-MS: 261 m/z (M^{+•}).

### Comparative Example 2a

### 5-[1,2]dithiolan-3-yl-pentanoic acid butylamide

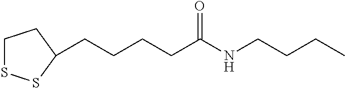

Yield: 75%

¹H-NMR (CDCl₃) 300 MHz: 5.70 (s, 1H), 3.57-3.48 (m, 1H), 3.23-3.05 (m, 4H), 2.47-2.37 (m, 1H), 2.16-1.77 (m, 3H), 1.70-1.20 (m, 10H), 0.88 (t, J=7.20, 3H); ¹³C-NMR (CDCl₃) 75 MHz: 172.6, 56.3, 40.1, 39.1, 38.4, 36.4, 34.5, 31.6, 28.8, 25.4, 20.0, 13.7; IR (CCl₄) ν_{N═H} 3465 cm⁻¹, ν_{C═O} 1662 cm⁻¹; GC-MS: 261 m/z (M^{+•}).

### Example 3

### (R)-5-[1,2]dithiolan-3-yl-pentanoic acid(3-morpholin-4-yl-propyl)-amide

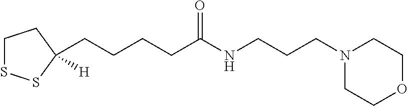

Yield: 60%

¹H-NMR (CDCl₃) 300 MHz: 7.27-6.80 (m, 1H), 6.9 (bs, 1H), 3.8-3.0 (m, 7H), 2.8-1.3 (m, 20H); IR (CHCl₃): ν_{N═H} 3446, 3317 cm⁻¹, ν_{C═O} 1654 cm⁻¹; GC-MS: 332 m/z (M^{+•}).

### Comparative Example 3a

### 5-[1,2]dithiolan-3-yl-pentanoic acid(3-morpholin-4-yl-propyl)-amide

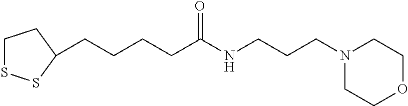

Yield: 55%

¹H-NMR (CDCl₃) 300 MHz: 7.27-6.80 (m, 1H), 6.9 (bs, 1H), 3.8-3.0 (m, 7H), 2.8-1.3 (m, 20H); IR (CHCl₃): ν_{N═H} 3446, 3317 cm⁻¹, ν_{C═O} 1654 cm⁻¹; GC-MS: 332 m/z (M^{30 •}).

### Example 4

### (R)-5-[1,2]dithiolan-3-yl-pentanoic acid(4-morpholin-4-yl-butyl)-amide

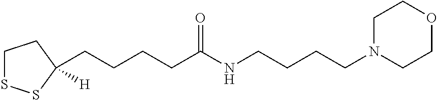

Yield: 57%

¹H-NMR (CDCl₃) 300 MHz: 5.93 (bs, 1H), 3.80-3.05 (m, 7H), 2.83-1.23 (m, 22H); ¹³C-NMR (CDCl₃) 75 MHz: 172.6, 66.9, 58.4, 56.5, 53.7, 40.3, 39.3, 38.5, 36.6, 34.7, 28.9, 27.5, 25.5, 24.0; IR (CHCl₃): ν_{N═H} 3448 cm⁻¹, ν_{C═O} 1660 cm⁻¹; GC-MS: 346 m/z (M^{+•}).

### Example 5

### (R)-5-[1,2]dithiolan-3-yl-pentanoic acid(1-methyl-butyl)-amide

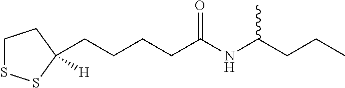

Yield: 40%

¹H-NMR (CDCl₃) 300 MHz: 5.38 (bs, 1H), 4.01-3.90 (m, 1H), 3.60-3.50 (sx, J=6.6, 1H), 3.20-3.06 (m, 2H), 2.50-1.81 (m, 4H), 1.78-1.22 (m, 10H), 1.08 (d, J=6.6, 3H), 0.88 (t, J=7.3, 3H); ¹³C-NMR (CDCl₃) 75 MHz: 171.9, 56.4, 44.9, 40.2, 39.1, 38.4, 36.6, 34.6, 28.8, 25.4, 21.0, 19.2, 13.9; IR (CHCl₃): ν_{N═H} 3437 cm⁻¹, ν_{C═O} 1664 cm⁻¹ GC-MS: 275 m/z (M^{+•}).

### Example 6

### (R)-5-[1,2]dithiolan-3-yl-pentanoic acid methylamide

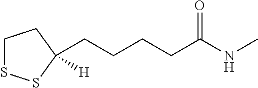

Yield: 35%

¹H-NMR (CDCl₃) 300 MHz: 5.70 (bs, 1H), 3.56-3.51 (m, 1H), 3.10-3.07 (m, 2H), 2.76 (d, J=4.6, 3H), 2.49-1.83 (m, 4H), 1.75-1.33 (m, 6H); ¹³C-NMR (CDCl₃) 75 MHz: 173.5, 56.4, 40.2, 38.4, 36.3, 34.6, 28.9, 26.3, 25.4; IR (CHCl₃): ν_{N═H} 3464 cm⁻¹, ν_{C═O} 1666 cm⁻¹; GC-MS: 219 m/z (M^{+•}).

### Example 7

### (R)-5-[1,2]dithiolan-3-yl-pentanoic acid propylamide

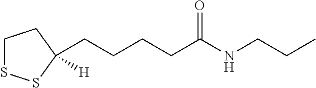

Yield: 40%

¹H-NMR (CDCl₃) 300 MHz: 5.42 (bs, 1H), 3.59-3.49 (m, 1H), 3.21-3.01 (m, 4H), 2.47-2.37 (m, 1H), 2.14 (t, J=7.3, 2H), 1.92-1.81 (m, 1H), 1.70-1.37 (m, 8H), 0.88 (t, J=7.5, 3H); ¹³C-NMR (CDCl₃) 75 MHz: 172.7, 56.4, 41.1, 40.2, 38.4, 36.5, 34.6, 28.8, 25.4, 22.8, 11.0; IR (CHCl₃): ν_{N═H} 3448 cm⁻¹, ν_{C═O} 1662 cm⁻¹; GC-MS: 247 m/z (M^{+•}).

### Example 8

### (R)-5-[1,2]dithiolan-3-yl-pentanoic acid isopropylamide

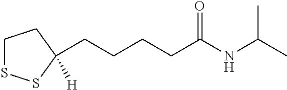

Yield: 30%

¹H-NMR (CDCl₃) 300 MHz: 5.35 (bs, 1H), 4.11-3.97 (m, 1H), 3.59-3.48 (sx, J=6.4, 1H), 3.20-3.07 (m, 2H), 2.48-2.38 (m, 1H), 2.11 (t, J=7.2, 2H), 1.97-1.82 (m, 1H), 1.74-1.33 (m, 6H), 1.11 (d, J=6.4, 6H); ¹³C-NMR (CDCl₃) 75 MHz: 171.7, 56.42, 41.2, 40.2, 38.4, 36.6, 34.6, 28.8, 25.4, 22.8; IR (CHCl₃): ν_{N═H} 3439 cm⁻¹, ν_{C═O} 1658 cm⁻¹; GC-MS: 247 m/z (M^{+•}).

### Example 9

### (R)-5-[1,2]dithiolan-3-yl-pentanoic acid(3-ethoxy-propyl)-amide

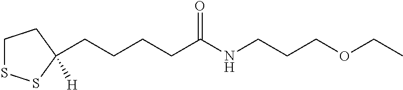

Yield: 60%

¹H-NMR (CDCl₃) 300 MHz: 6.20-6.90 (m, 1H), 3.56-2.58 (m, 9H), 2.48-2.30 (m, 1H), 2.09 (t, J=7.3, 2H), 1.92-1.24 (m, 9H), 1.13 (t, J=7.0 , 3H); IR (CHCl₃): ν_{N═H} 3450, 3406 cm⁻¹, ν_{C═O} 1660, 1661 cm⁻¹; GC-MS: 291 m/z (M^{+•}).

### Example 10

### (R)-5-[1,2]dithiolan-3-yl-pentanoic acid(2-acetylamino-ethyl)-amide

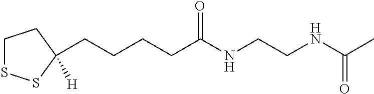

Yield: 55%

¹H-NMR (CDCl₃CD₃OD) 300 MHz: 3.90-3.81 (m, 1H), 3.47-2.91 (m, 6H), 2.37-2.20 (m, 1H), 2.00 (t, J=7.3, 2H), 1.90-1.20 (m, 10H); ¹³C-NMR (CDCl₃CD₃OD) 75 MHz: 174.5, 172.0, 56.1, 39.9, 39.0, 38.9, 38.1, 35.7, 34.2, 28.5, 25.0, 22.2; IR (CHCl₃): ν_{N═H} 3452 cm⁻¹, ν_{C═O} 1660 cm⁻¹; m.p.: 144.6-145.8° C.; GC-MS: 290 m/z (M^{+•}).

### Comparative Example 10a

### 5-[1,2]dithiolan-3-yl-pentanoic acid(2-acetylamino-ethyl)-amide

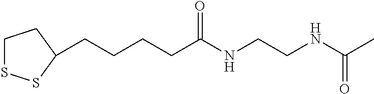

Yield: 50%

¹H-NMR (CDCl₃CD₃OD) 300 MHz: 3.90-3.81 (m, 1H), 3.47-2.91 (m, 6H), 2.37-2.20 (m, 1H), 2.00 (t, J=7.3, 2H), 1.90-1.20 (m, 10H); ¹³C-NMR (CDCl₃CD₃OD) 75 MHz: 174.5, 172.0, 56.1, 39.9, 39.0, 38.9, 38.1, 35.7, 34.2, 28.5, 25.0, 22.2; IR (CHCl₃): ν_{N═H} 3452 cm⁻¹, ν_{C═O} 1660 cm⁻¹; m.p.: 144.6-145.8° C.; GC-MS: 290 m/z (M^{+•}).

### Example 11

### (R)-5-[1,2]dithiolan-3-yl-pentanoic acid phenethyl-amide

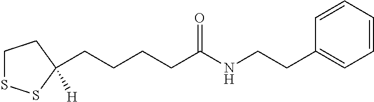

Yield: 32%

¹H-NMR (CDCl₃) 300 MHz: 7.32-7.13 (m, 5H), 5.52 (bs, 1H), 3.56-3.03 (m, 5H), 2.82-2.70 (m, 2H), 2.47-2.35 (m, 1H), 2.10 (t, J=7.2, 2H), 1.92-1.81 (m, 1H), 1.68-1.33 (m, 6H); ¹³C-NMR (CDCl₃) 75 MHz: 172.7, 138.8, 128.7, 128.6, 126.5, 56.4, 40.5, 40.2, 38.4, 36.4, 35.6, 34.6, 28.8, 25.3; IR (CHCl₃): ν_{N═H} 3448 cm⁻¹, ν_{C═O} 1662 cm⁻¹; GC-MS: 309 m/z (M^{+•}).

### Example 12

### (R)-5-[1,2]dithiolan-3-yl-pentanoic acid(3-phenyl-propyl)-amide

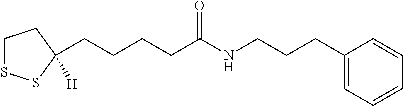

is Yield: 35%

¹H-NMR (CDCl₃) 300 MHz: 7.29-7.12 (m, 5H), 5.61 (bs, 1H), 3.58-3.47 (m, 1H), 3.28-3.00 (m, 4H), 2.63 (t, J=7.6, 2H), 2.47-2.36 (m, 1H), 2.11 (t, J=7.3, 2H), 1.91-1.38 (m, 9H); ¹³C-NMR (CDCl₃) 75 MHz: 172.6, 141.6, 128.4, 128.2, 125.9, 56.3, 40.1, 39.1, 38.4, 36.4, 34.5, 33.2, 31.1, 28.8, 25.3; IR (CHCl₃): ν_{N═H} 3448 cm⁻¹, ν_{C═O} 1662 cm⁻¹; GC-MS: 323 m/z (M^{+•}).

### Example 13

### (R)-5-[1,2]dithiolan-3-yl-pentanoic acid(4-phenyl-butyl)-amide

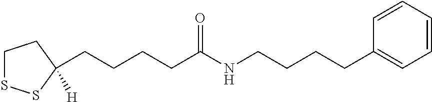

Yield: 30%

¹H-NMR (CDCl₃) 300 MHz: 7.28-7.10 (m, 5H), 5.65 (bs, 1H), 3.60-3.48 (m, 1H), 3.29-3.02 (m, 4H), 2.60 (t, J=7.6, 2H), 2.43-2.38 (m, 1H), 2.12 (t, J=7.3, 2H), 1.91-1.78 (m, 1H), 1.69-1.36 (m, 10H); ¹³C-NMR (CDCl₃) 75 MHz: 172.6, 142.0, 128.3, 128.2, 125.7, 56.3, 40.1, 39.2, 38.4, 36.4, 35.4, 34.5, 29.1, 28.8, 28.6, 25.4; IR (CHCl₃): ν_{N═H} 3448 cm⁻¹, ν_{C═O} 1662 cm⁻¹; GC-MS: 337 m/z (M^{+•}).

### Example 14

### (R)-5-[1,2]dithiolan-3-yl-pentanoic acid cyclopentylamide

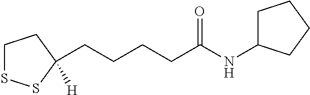

Yield: 20%

¹H-NMR (CDCl₃) 300 MHz: 5.72 (bs, 1H), 4.19-4.06 (m, 1H), 3.59-3.47 (m, 1H), 3.17-3.00 (m, 2H), 2.48-2.33 (m, 1H), 2.09 (t, J=7.3, 2H), 1.99-1.23 (m, 15H); ¹³C-NMR (CDCl₃) 75 MHz: 172.2, 56.3, 50.9, 40.1, 38.4, 36.4, 34.5, 33.0, 28.8, 25.4, 23.6; IR (CHCl₃): ν_{N—H} 3441 cm⁻¹, ν_{C═O} 1654 cm⁻¹; GC-MS: 273 m/z (M^{+•}).

### Example 19

### (R)-5-[1,2]dithiolan-3-yl-pentanoic acid n-pentylamide

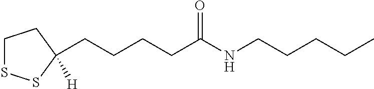

Yield: 30%

¹H-NMR (CDCl₃) 300 MHz: 0.55 (bs, 1H), 3.63-3.47 (m, 1H), 3.27-3.06 (m, 4H), 2.52-2.36 (m, 1H), 2.15 (t, J=7.2, 2H), 1.97-1.73 (m, 1H), 1.71-1.20 (m, 12H), 0.86 (t, J=6.2, 3H); ¹³C-NMR (CDCl₃) 75 MHz: 172.4, 56.2, 40.0, 39.2, 38.2, 36.3, 34.3, 29.1, 28.8, 28.6, 25.2, 22.1, 13.7; IR (CHCl₃): ν_{N═H} 3448 cm⁻¹, ν_{C═O} 1660cm⁻¹; GC-MS: 275 m/z (M^{+•}).

### Example 20

### (R)-5-[1,2]dithiolan-3-yl-pentanoic acid n-heptylamide

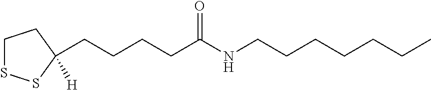

Yield: 40%

¹H-NMR (CDCl₃) 300 MHz: 5.61 (bs, 1H), 3.61-3.48 (m, 1H), 3.26-3.04 (m, 4H), 2.51-2.35 (m, 1H), 2.14 (t, J=7.3, 2H), 1.96-1.72 (m, 1H), 1.70-1.15 (m, 16H), 0.85 (t, J=6.1, 3H); ¹³C-NMR (CDCl₃) 75 MHz: 172.6, 56.4, 40.2, 39.5, 38.5, 36.6, 34.6, 31.7, 29.7, 29.0, 28.9, 26.9, 25.4, 22.6, 14.0; IR (CHCl₃): ν_{N═H} 3446 cm⁻¹, ν_{C═O} 1662 cm⁻¹; GC-MS: 303 m/z (M^{+•}).

### Example 21

### (R)-5-[1,2]dithiolan-3-yl-pentanoic acid pentan-3-ylamide

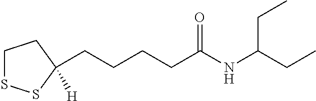

Yield: 31%

¹H-NMR (CDCl₃) 300 MHz: 5.40-5.32 (m, 1H), 3.81-3.66 (m, 1H), 3.57-3.46 (m, 1H), 3.02-3.01 (m, 2H), 2.49-2.35-(m, 1H), 2.15 (t, J=7.4, 2H), 1.93-1.81 (m, 1H), 1.75-1.22 (m, 10H), (0.85, t, J=7.4, 6H); ¹³C-NMR (CDCl₃) 75 MHz: 172.3, 56.3, 51.7, 40.1, 38.4, 36.7, 34.6, 28.8, 27.4, 25.5, 10.2; IR (CHCl₃): ν_{N═H} 3433 cm⁻¹, ν_{C═O} 1654 cm⁻¹; GC-MS: 275 m/z (M^{+•}).

### Example 22

### (R)-5-[1,2]dithiolan-3-yl-pentanoic acid N-benzylamide

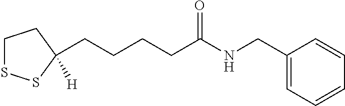

Yield: 32%

¹H-NMR (CDCl₃) 300 MHz: 7.33-7.23 (m, 5H), 5.97 (bs, 1H), 3.02(d, J=5.9, 2H), 3.57-3.48 (m, 1H), 3.19-3.03 (m, 2H), 2.47-2.36 (m, 1H), 2.19 (t, J=7.3, 2H), 1.92-1.81 (m, 1H), 1.74-1.35 (m, 6H); ¹³C-NMR (CDCl₃) 75 MHz: 172.5, 138.3, 128.6, 127.7, 127.4, 56.3, 43.5, 40.1, 38.4, 36.3, 34.5, 28.8, 25.3; IR (CHCl₃): ν_{N═H} 3444 cm⁻¹, ν_{C═O} 1662 cm⁻¹; GC-MS: 295 m/z (M^{+•}).

### Example 23

### (R)-5-[1,2]dithiolan-3-yl-pentanoic acid N-(3-hydroxy-4methoxybenzyl)amide

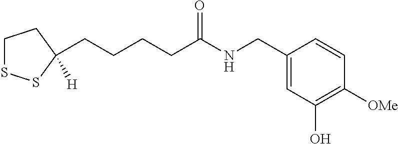

Yield: 51%

¹H-NMR (CDCl₃) 300 MHz: 6.88-6.70 (m, 3H), 6.05-5.91 (m, 2H), 4.29 (d, J=5.5, 2H), 3.85 (s, 3H), 3.63-3.47 (m, 1H), 3.22-3.04 (m, 2H), 2.50-2.37 (m, 1H), 2.18 (t, J=7.7, 2H), 1.97-1.80 (m, 1H), 1.77-1.33 (m, 7H); ¹³C-NMR (CDCl₃) 75 MHz: 172.4, 145.9, 145.7, 131.3, 119.3, 114.0, 110.6, 56.2, 55.8, 43.0, 40.0, 38.3, 36.2, 34.4, 28.7, 25.2; IR (CHCl₃): ν_{N═H} 3545 cm⁻¹, ν_{C═O} 1662 cm⁻¹; GC-MS: 341 m/z (M^{+•}).

### Example 24

### (R)-5-[1,2]dithiolan-3-yl-pentanoic acid N-(cyclopropylmethyl)amide

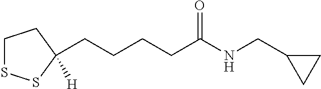

Yield: 42%

¹H-NMR (CDCl₃) 300 MHz: 5.77 (bs, 1H), 3.63-3.45 (m, 1H), 3.24-2.96 (m, 4H), 2.52-2.34 (m, 1H), 2.16 (t, J=7.3, 2H), 1.93-1.82 (m, 1H), 1.75-1.33 (m, 6H), 0.98-0.84 (m, 1H), 0.53-0.41 (m, 2 H), 0.21-0.10 (m, 2H); ¹³C-NMR (CDCl₃) 75 MHz: 172.4, 56.2, 44.1, 40.0, 38.3, 36.3, 34.4, 28.7, 25.3, 10.5, 3.2; IR (CHCl₃): ν_{N═H} 3448 cm⁻¹, ν_{C═O} 1660 cm⁻¹; GC-MS: 259 m/z (M^{+•}).

### Example 25

### (R)-5-[1,2]dithiolan-3-yl-pentanoic acid N-(buten-3-yl)amide

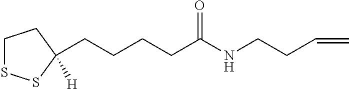

Yield: 45%

¹H-NMR (CDCl₃) 300 MHz: 5.82-5.62 (m, 2H), 5.12-4.98 (m, 2H), 3.60-3.48 (m, 1H), 3.35-3.25 (m, 2H), 3.18-3.01 (m, 2H), 2.49-2.34 (m, 1H), 2.25-2.18 (m, 2H), 2.13 (t, J=7.3, 2H), 1.92-1.81 (m, 1H), 1.72-1.33 (m, 6H); ¹³C-NMR (CDCl₃) 75 MHz: 172.5, 135.1, 116.9, 56.2, 40.0, 38.3, 38.2, 36.3, 34.4, 33.6, 28.7, 25.2; IR (CHCl₃): ν_{N═H} 3446 cm⁻¹, ν_{C═O} 2252 cm⁻¹, ν_{C═O} 1662 cm⁻¹; GC-MS: 259 m/z (M^{+•}).

### Example 26

### (R)-5-[1,2]dithiolan-3-yl-pentanoic acid 1-(4-acetylpiperazin-1-yl)amide

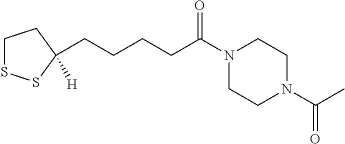

Yield: 37%

¹H-NMR (CDCl₃) 300 MHz: 3.66-3.40 (m, 9H), 3.21-3.00 (m, 2H), 2.49-2.33 (m, 1H), 2.31 (t, J=7.7, 2H), 2.07 (s, 3H), 1.94-1.79 (m, 1H), 1.74-1.34 (m, 6H); ¹³C-NMR (CDCl₃) 75 MHz: 171.3, 169.1, 56.2, 45.8, 44.9, 41.3, 41.2, 40.0, 38.3, 34.5, 32.7, 28.8, 24.6, 21.2; IR (CHCl₃): ν_{C═O} 1637 cm⁻¹; GC-MS: 316 m/z (M^{+•}).

### Example 27

### (R)-5-[1,2]dithiolan-3-yl-pentanoic acid N-(3-acetoxy-4methoxybenzyl)amide

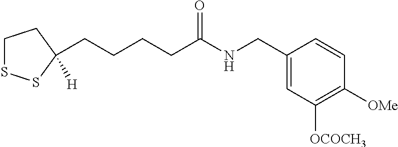

Yield: 70%

¹H-NMR (CDCl₃) 300 MHz: 7.12-7.07 (m, 1H), 6.94-6.88 (m, 2H), 5.89 (bs, 1H), 4.31 (d, J=5.9, 2H), 3.80 (s, 3H), 3.62-3.48 (m, 1H), 3.22-3.04 (m, 2H), 2.53-2.37 (m, 1H), 2.28 (s, 3H), 2.17 (t, J=7.3, 2H), 1.97-1.31 (m, 7H); ¹³C-NMR (CDCl₃) 75 MHz: 172.4, 168.9, 150.3, 139.5, 130.8, 136.3, 122.3, 112.3, 56.2, 55.8, 42.6, 40.0, 38.3, 36.2, 34.4, 28.7, 25.2, 20.5; IR (CHCl₃): ν_{N═H} 3444cm⁻¹, ν_{C═O} 1763 cm⁻¹, ν_{C═O} 1668 cm⁻¹; GC-MS: 383 m/z (M^{+•}).

### Example 28

### (R)-5-[1,2]dithiolan-3-yl-pentanoic acid 1-(4-methylpiperidin-1-yl)amide

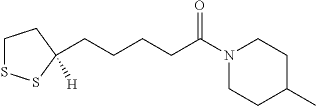

Yield: 60%

¹H-NMR (CDCl₃) 300 MHz: 4.58-4.52 (m, 1H), 3.81-3.76 (m, 1H), 3.61-3.52 (m, 1H), 3.21-3.05 (m, 2H), 3.01-2.91 (m, 1H), 2.55-2.39 (m, 2H), 2.31 (t, J=7.5, 2H), 1.95-1.84 (m, 1H), 1.78-1.38 (m, 9H), 1.11-0.99 (m, 2H), 0.93 (d, J=6.4, 3H); ¹³C-NMR (CDCl₃) 75 MHz: 170.9, 56.4, 45.9, 41.9, 40.2, 38.4, 34.7, 34.6, 33.7, 33.1, 31.0, 29.1, 25.0, 21.7; IR (CHCl₃): ν_{C═O} 1643 cm⁻¹; GC-MS: 287 m/z (M^{+•}).

### Preparation of the compounds of Examples 15-18

A two-neck round-bottom flask equipped with magnetic stirrer was flame-heated under argon flow then covered with silver paper to avoid exposure to light. A solution of (RS) alpha-lipoic acid (Examples 15a, 16a) or (R) alpha-lipoic acid (Examples 15-18) (1 mole) in DMF was then prepared. The solution was stirred and then K₂CO₃ (1.15 mols) was added. The resulting mixture was maintained at room temperature under argon atmosphere with stirring for about 2 hours. After this time, the halide (1.2 mols) was added and the reaction mixture left at room temperature with stirring until TLC analysis (SiO₂, MeOH/CHCl₃: 5/95, Et₂O/hexane: 1/1) showed the disappearance of the starting product as also confirmed by GC-MS analysis.

The reaction mixture was then transferred into a separating funnel, having taken care to previously cover the glassware to be used with silver paper to avoid exposing the solution to light. After washing with saline, the aqueous phase was extracted with Et₂O (4×10 ml) and the pooled organic phases were dried over anhydrous Na₂SO₄, filtered and evaporated under reduced pressure. The mixture thus obtained was then transferred to a dark coloured flask and kept at a high vacuum suction pump (24 hours) to remove DMF. The mixture was then purified over a chromatography column (SiO₂, Et₂O/n-hexane: 1/9). The alpha-lipoic acid derivative compound was then separated and characterized by GC-MS analysis and ¹H-NMR, ¹³C-NMR and IR spectroscopy.

### Example 15

### (R)-5-[1,2]dithiolan-3-yl-pentanoic acid 2,2-dimethyl-propionyloxymethyl ester

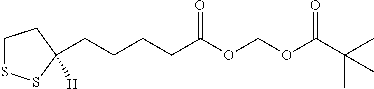

Yield: 45%

¹H-NMR (CDCl₃) 300 MHz: 5.73 (s, 2H), 3.60-3.47 (m, 1H), 3.21-3.03 (m, 2H), 2.50-2.37 (m, 1H), 2.35 (t, J=7.3, 2H), 1.95-1.81 (m, 1H), 1.74-1.38 (m, 6H), 1.18 (s, 9H); ¹³C-NMR (CDCl₃) 75 MHz: 176.9, 171.9, 79.2, 50.0, 40.1, 38.9, 38.3, 34.4, 33.5, 28.4, 26.7, 24.2; IR (CCl₄) ν_{C═O} 1757, 1749 cm⁻¹; GC-MS: 320 m/z (M^{+•}).

### Comparative Example 15a

### 5-[1,2]dithiolan-3-yl-pentanoic acid 2,2-dimethyl-propionyloxymethyl ester

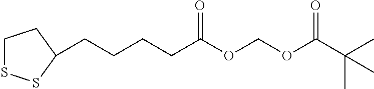

Yield: 73%

¹H-NMR (CDCl₃) 300 MHz: 5.73 (s, 2H), 3.59-3.50 (m, 1H), 3.20-3.05 (m, 2H), 2.49-2.33 (m, 3H), 1.94-1.83 (m, 1H), 1.68-1.63 (m, 4H), 1.51-1.41 (m, 2H), 1.94 (s, 9H); ¹³C-NMR (CDCl₃) 75 MHz: 171.7, 172.0, 79.3, 56.2, 40.1, 38.7, 38.4, 34.5, 33.7, 28.5, 26.8, 24.3; IR (CCl₄) ν_{C═O} 1746 cm⁻¹; GC-MS: 320 m/z (M^{+•}).

### Example 16

### (R)-5-[1,2]dithiolan-3-yl-pentanoic acid butyryloxymethyl ester

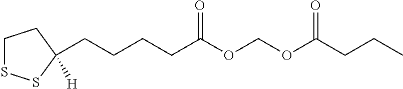

Yield: 40%

¹H-NMR (CDCl₃) 300 MHz: 5.72 (s, 2H), 3.62-3.47 (m, 1H), 3.23-3.03 (m, 2H), 2.50-2.28 (m, 5H), 1.95-1.80 (m, 1H), 1.73-1.34 (m, 8H), 0.93 (t, J=7.3, 3H); ¹³C-NMR (CDCl₃) 75 MHz: 172.1, 171.9, 78.9, 56.1, 40.0, 38.3, 35.6, 34.3, 33.5, 28.4, 24.1, 17.9, 13.3; IR (CCl₄) ν_{C═O} 1751 cm⁻¹; GC-MS: 306 m/z (M⁺).

### Comparative Example 16a

### 5-[1,2]dithiolan-3-yl-pentanoic acid butyryloxymethyl ester

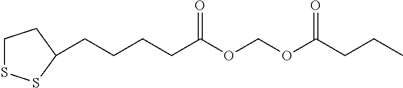

Yield: 63%

¹H-NMR (CDCl₃) 300 MHz: 5.69(s, 2H), 3.55-3.46 (m, 1H), 3.17-3.01 (m, 2H), 2.46-2.26 (m, 5H), 1.91-1.79 (m, 1H), 1.68-1.38 (m, 8H), 0.89 (t, J=7.4, 3H); ¹³C-NMR (CDCl₃) 75 MHz: 172.0, 171.9, 78.8, 56.1, 40.0, 38.3, 35.6, 34.3, 33.5, 28.4, 24.1, 17.9, 13.4; IR (CCl₄) ν_{C═O} 1752 cm⁻¹; GC-MS: 306 m/z (M⁺).

### Example 17

### (R)-5-[1,2]dithiolan-3-yl-pentanoic acid 1-acetoxy-ethyl ester

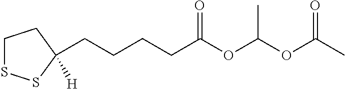

Yield: 35%

¹H-NMR (CDCl₃) 300 MHz: 6.81 (q, J=5.4, 1H), 3.59-3.47 (m, 1H), 3.20-3.02 (m, 2H), 2.50-2.36 (m, 1H), 2.29 (t, J=7.2, 2H), 2.03 (s, 3H), 1.94-1.80 (m, 1H), 1.74-1.55 (m, 6H), 1.43 (d, J=5.4, 3H); ¹³C-NMR (CDCl₃) 75 MHz: 171.3, 168.8, 88.4, 56.2, 40.1, 38.4, 34.5, 33.7, 28.5, 24.2, 20.8, 19.5; IR (CHCl₃): ν_{C═O} 1751, 1751 cm⁻¹; GC-MS: 292 m/z (M^{+•}).

### Example 18

### (R)-5-[1,2]dithiolan-3-yl-pentanoic acid 2,2-dimethyl-propionyloxyethyl ester

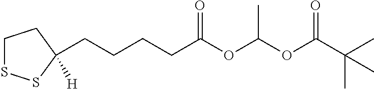

Yield: 42%

¹H-NMR (CDCl₃) 300 MHz: 6.80 (q, J=5.5, 1H), 3.60-3.48 (m, 1H), 3.22-3.03 (m, 2H), 2.50-2.38 (m, 1H), 2.31 (t, J=7.3, 2H), 1.97-1.83 (m, 1H), 1.75-1.46 (m, 6H), 1.44 (d, J=5.5, 3H), 1.18 (s, 9H); ¹³C-NMR (CDCl₃) 75 MHz: 176.4, 171.4, 88.5, 56.2, 40.2, 38.6, 38.5. 34.5, 33.8, 28.5, 26.8, 24.4, 19.4; IR (CHCl₃): ν_{C═O} 1749 cm⁻¹, ν_{C═O} 1732 cm⁻¹; GC-MS: 336 m/z (M^{+•}).

### Example 29

Evaluation of Cell Viability

Dose-dependent concentration studies of the synthesized compounds were carried out and compared with the effects of alpha-lipoic acid, defined as reference standard, on human hepatocarcinoma cells (HepG2 cells) with the aim of evaluating their effects on cell viability.

Analysis by optical microscope enabled any morphological changes and accumulation of lipid vesicles to be determined as well as to evaluate cell proliferation or arrest.

A luminometric assay (ATPlite, Perkin Elmer), based on the production of light caused by a reaction with the intracellular ATP of viable cells, enabled the compounds to be classified on the basis of their viability index. In this respect, the signal detected by the luminometer is proportional to the number of viable cells.

Methodology
HepG2 cells were seeded in sterile 96-well plates, at a concentration of 5×10³ cells/well in 100 μl of culture medium (RPMI supplemented with 10% fetal bovine serum (FBS) and enriched with 2 mM of glutamine, 200 U/ml penicillin and 200 U/ml streptomycin).
The plates thus obtained were incubated at 37° C. at 5% CO₂.
The compounds of the invention, including alpha-lipoic acid were dissolved in dimethyl sulphoxide (DMSO) at an initial concentration of 100 mM then diluted directly in the culture medium until the following concentrations were attained: 5, 10, 100, 500 and 1000 μM.
After 16-24 hours from seeding, the medium was renewed with 100 μl of RPMI+10% FBS containing the specific concentration of the compound of the invention and the corresponding concentration of DMSO as control.
The culture medium containing the suitable concentration of the compound of the invention was renewed every 24 hours and, after two treatments, a qualitative and quantitative analysis was carried out on the effects of the compounds under study. Using the optical microscope, changes were observed in the number of adherent cells and the morphology (data not given), while the viability was evaluated by using reagents and methods described in the ATPlite kit, marketed by Perkin Elmer.

Results

The results obtained after treatment for 48 hours with 500 μM of α-LA and 2 compounds of the invention, either in racemic form (RS) (Comparative Examples 2a and 10a) or in enantiomeric form (R) (Examples 2 and 10) are given in FIG. 1, indicated as cell viability compared to the control (DMSO 0.5%) (in FIG. 1, * refers to (RS)-α-LA and (R)-α-LA purchased from Sigma-Aldrich).

For all the tested compounds, it was observed that the enantiomer R was responsible for a greater cell viability index than the racemic form RS. This allowed to confirm that the enantiomers R of the compounds of formula I, similarly to alpha-lipoic acid as such, were significantly less toxic and pharmacologically advantageously more bioavailable than the corresponding racemic form.

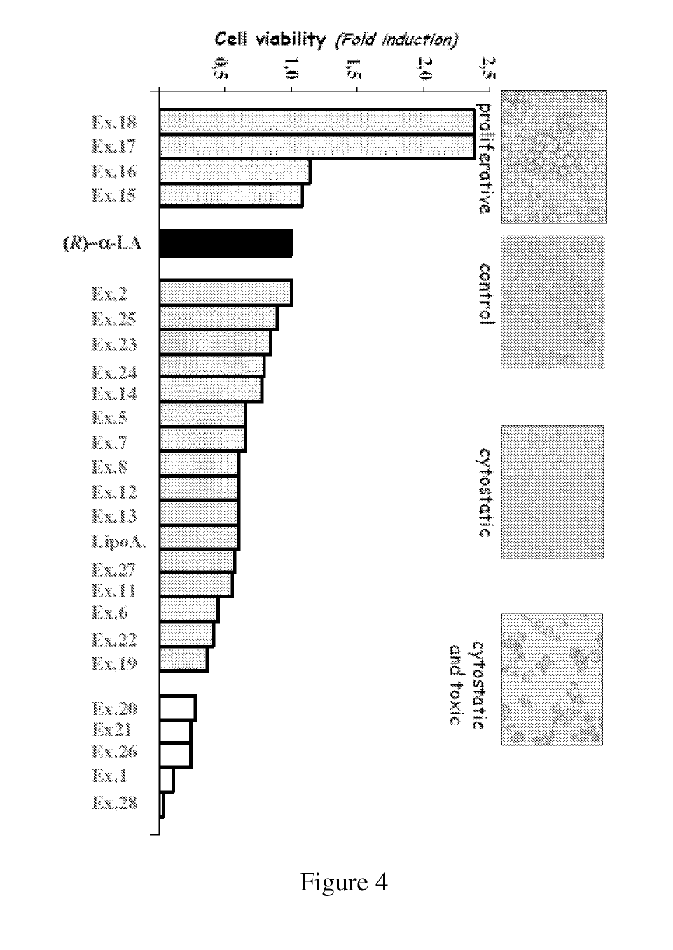

FIG. 4 gives cell viability results relating to treatment with the enantiomers of the invention compared to those obtained after treatment with (R)-α-LA at a 1 mM concentration and with lipoamide (lipoA., from Sigma-Aldrich) at a 1 mM concentration. This concentration was selected as the reference parameter because under these conditions, (R)-α-LA presented a cell viability of 50% compared to the control (1% DMSO). Cell viability is given as “fold induction” of luminescence correlated to intracellular ATP following treatment with the compound of the invention relative to administration of (R)-α-LA (unit value). Treatment with (R)-α-LA resulted in arrest of cell proliferation, in that, unlike the control, the HepG2 cells were unable to colonize all the culture plate and reached 60-70% confluence (data not given).

Viability and cell proliferation data hence enabled the enantiomers under analysis to be subdivided as follows:

a) Proliferative: enantiomers showing a greater viability index than (R)-α-LA, with a even multi-layered proliferation, a physiological accumulation of lipid vesicles and an absence of cells in suspension; the enantiomers belonging to this group are those of Examples 18, 17, 16, 15, listed in decreasing order, according to FIG. 4;

b) Cytostatic: enantiomers showing a lower viability index than that shown by (R)α-LA, but at the same time being potent inducers of cell proliferation arrest; the enantiomers belonging to this group are those of Examples 2, 25, 23, 24, 14, 5, 7, 8, 12, 13, lipoA. (i.e. lipoamide, as comparative compound), Examples 27, 11, 6, 22, 19, listed in decreasing order, according to FIG. 4;

and

c) Cytotoxic: enantiomers causing a significantly high number of cells in suspension, which is a clear evidence of cell death occurred; the enantiomers belonging to this group are those of Examples 20, 21, 26, 1, 28, listed in decreasing order, according to FIG. 4.

### Example 30

In Vitro Enzyme Hydrolysis Studies

In view of what found from the results of Example 29, an in vitro enzyme hydrolysis assay was carried out with the aim of further clarifying which of the synthesized derivatives behaved as alpha-lipoic acid precursors, i.e. as prodrugs able to release alpha-lipoic acid by hydrolysis. Differently, the derivatives that did not undergo hydrolysis were regarded as potential α-LA analogues.

A protein pool extracted from murine liver, containing enzymes able to promote hydrolysis of the compounds of the invention, was used to reintroduce in vitro the hydrolytic activity previously described in the pharmacokinetic studies.

Methodology
The liver of a wild-type mouse was removed; excess blood was removed by washing in phosphate buffer saline (PBS);
Said liver was transferred into a homogenizer with a sufficient amount of lysis buffer (50 mM Tris HCl pH 7.5-8.0; 150 mM NaCl; 5 mM EGTA pH 7.5-8.0; 50 mM NaF pH 8.0; 10% (v/v) glycerol; 1.5 mM MgCl₂, 1% Triton X-100) enriched with protease and phosphatase inhibitors (Protease Inhibitor Cocktail; Sigma-Aldrich). 15-20 impacts were applied by the pestle in order to obtain a homogeneous solution;
The entire mixture was left under agitation at +4° C. for 1 hour; after centrifugation at 14000 rpm, the supernatant containing the extracted proteins was recovered;
The amount of extracted protein was determined using the Bradford assay;
The enzymatic hydrolysis reaction was carried out: 1 mg of protein extract for hydrolysing 50 μg of synthesized compound (dissolved in DMSO); 1, 3 or 24 hours at 25° C. under agitation;
The digestion product(s) was/were detected using thin-layer chromatography: mobile phase (60:40:1 of ethyl acetate; n-hexane; acetic acid); silica plate (Sigma-Aldrich); phosphomolybdic reagent;
The digestion products were analyzed and compared with (R)-α-LA (Sigma-Aldrich), and with the non-hydrolysed compound of the invention.

Results

Under the above described experimental conditions for thin-layer chromatography, the synthesized enantiomers of the invention were identified as having a different “Ratio frontis” (R_{f}) from that of (R)-alpha-lipoic acid. It was, therefore, possible to detect (R)-alpha-lipoic acid as the hydrolysis product and to differentiate it from the enantiomers of the invention.

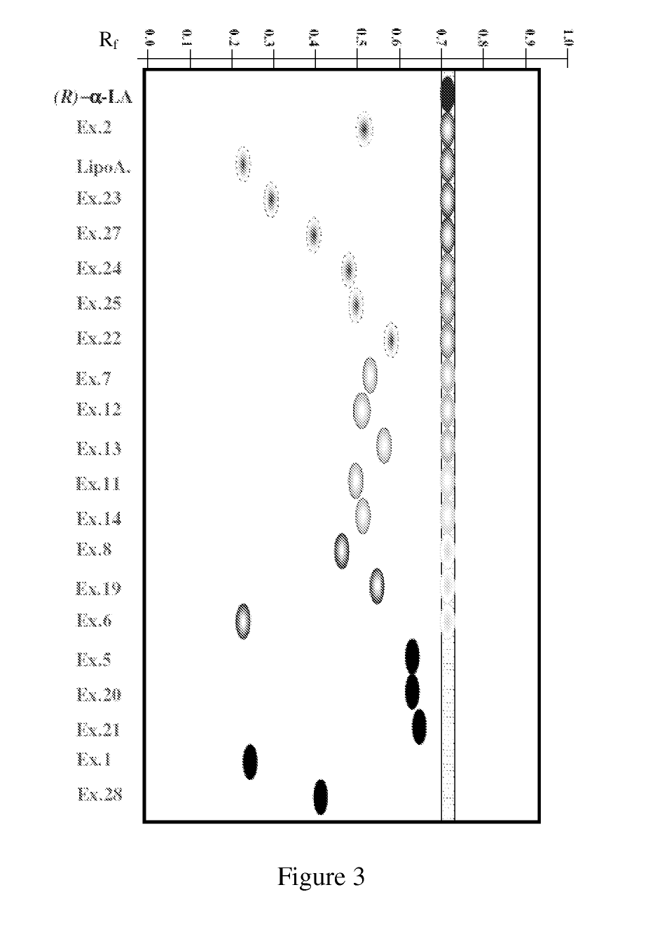

FIGS. 2 and 3 show an image of the enzymatic digestion products obtained after the chromatographic run. The enantiomers under analysis were found to differ in the efficiency of enzymatic hydrolysis, and consequently in the amounts of released (R)-α-LA.

Particularly, FIG. 2 refers to the enantiomers of formula II, specifically to Examples 15-18 included therein, and shows the digestion results after 1h and after 3 h for all these 4 enantiomers. The enantiomers of Examples 15 and 16 were completely hydrolyzed after 1 hours, as it can be seen by the intensity of the stains concerned at R_{f}=0.7, corresponding to the r_{f} of the (R)-alpha-lipoic acid as such. Differently, the Examples 17 and 18 showed a partial hydrolization after 1 hour, thus indicating that these two enantiomers released (R)-alpha-lipoic acid more slowly than the Examples 15 and 16. After 3 hours, also the enantiomers of the Examples 17 and 18 were completely hydrolyzed.

These results allowed to consider that the enantiomers of the Examples 15, 16, 17 and 18 could be successfully used as prodrugs able to release (R)-alpha-lipoic acid.

FIG. 3 refers to the enantiomers as synthesized in the above Examples, and shows the digestion results after 24 hours at 25° C. Even in this case, the intensity of the stains is directly proportional to the detected amount of the (R)-alpha-lipoic acid and the enantiomers concerned.

It should be noted that the enantiomers in FIG. 3 have been listed in accordance with their ability to release (R)-alpha-lipoic acid, so by displaying them in a decreasing order. Particularly, the enantiomers of Examples 2, 23, 27, 24, 25 and 22, even if not completely hydrolysed after 24 hours, have proved to release a very considerable amount of (R)-alpha-lipoic acid. Therefore, these enantiomers could be advantageously used as prodrugs of (R)-alpha-lipoic acid also in slow release formulations.

The Examples 7, 12, 13, 11, 14, 8, 19 and 6 showed a partial hydrolization after 24 hours and consequently a reduced release of (R)-alpha-lipoic acid; therefore these enantiomers could be successfully used as prodrugs or as analogues of (R)-alpha-lipoic acid.

The Examples 5, 20, 21, 1 and 28 remained essentially unaltered after 24 hours, thus these enantiomers could be conveniently used as analogues of (R)-alpha-lipoic acid. It should be noted that, in view of the results of Example 29, the enantiomers of Examples 20, 21, 1 and 28 could find a very appreciable application as analogues of (R)-alpha-lipoic acid in the treatment of tumours.

### Example 31

Pharmacokinetic Studies

The bioavailability of some compounds synthesized (Examples 1a, 2, 2a, 3, 3a, 10, 15a, 16a and 24) was studied in rats after oral administration of a single dose (48.5 μmol/kg) and compared with the reference (R)- or (RS)-α-LA.

Methodology

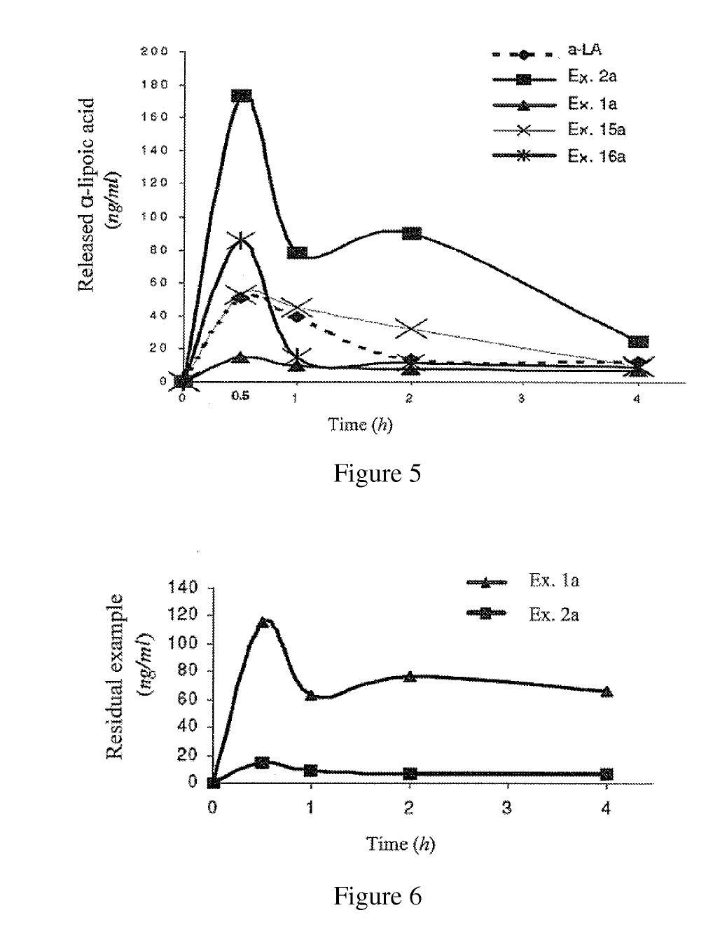

Each treatment was carried out on groups of 6 male rats (Sprague-Dawley SD rat) per compound. At regular times, blood withdrawals were carried out for the measurement by HPLC-MS of the concentration of plasmatic α-LA (FIGS. 5, 7, 9) and of the residual compound administered (FIGS. 6, 8).

Results

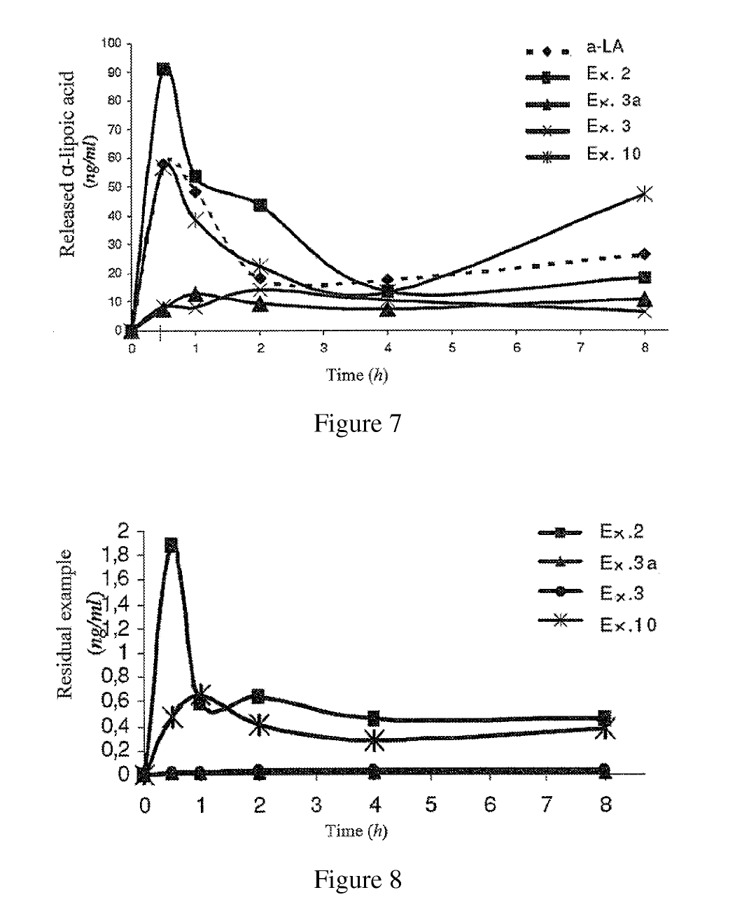

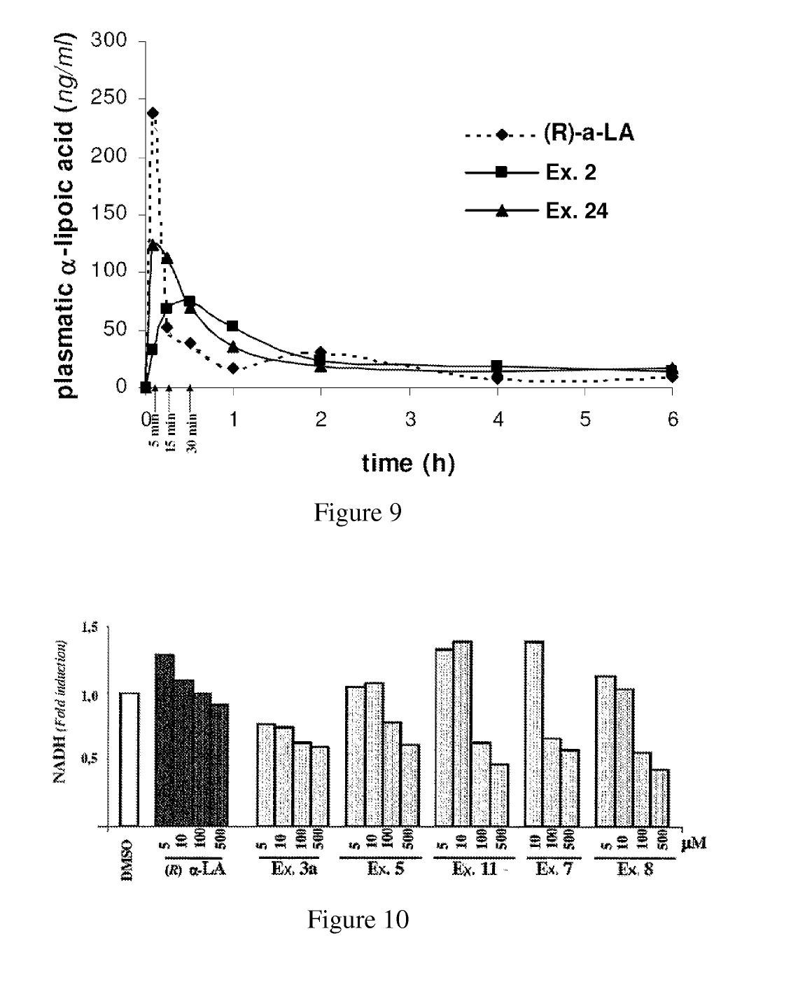

Plasma measurements of concentration α-LA were carried out for the compounds of Comparative Examples 1a, 2a, 15a, 16a (FIG. 5), Examples 2, 3, 3a and 10 (FIG. 7) and Examples 2 and 24 (FIG. 9). The analysis was performed at the time (hours) indicated after oral administration of compounds or reference and for this series of Examples the maximum plasma concentration of released α-LA was also found to be at 30 minutes. The enantiomer R of Example 2 (FIG. 7) exhibited a more linear hydrolysis profile of the corresponding racemic compound (FIG. 5). The data shown in FIGS. 6 and 8 relating to the amount of non-hydrolysed compound (<<2 ng/ml) allowed Examples 2, 2a and 10 to be classified as alpha-lipoic acid prodrugs. Finally, for the enantiomer R of Example 3 and the corresponding racemic compound of the Comparative Example 3a, neither released alpha-lipoic acid nor the compound itself was detected for any of the withdrawal times.

In order to further clarify the pharmacokinetics, an additional analysis of plasmatic (R)-α-LA concentration after oral administration of two compounds (Example 2 and 24) was carried out for shorter treatment intervals (5-30 minutes) (FIG. 9). It was confirmed that, after 30 minutes from administration of the enantiomer R of Example 2, a significant concentration of alpha-lipoic acid in plasma was observed. In contrast, for the enantiomer R of Example 24 (and for exogenous alpha-lipoic acid used as reference) only 5 minutes of treatment were enough for detecting a peak of plasmatic (R)-α-LA plasma.

### Example 32

Study of Variation in NADH and NADPH Levels in the Presence of α-LA and the Compounds of the Present Invention

Although it is known that under hyperglycemic conditions α-LA induces AMPK activation in hepatic cells, the molecular mechanisms responsible for this biochemical process are not yet known. As envisaged in the description, the inventors of the present invention have advanced the hypothesis that AMPK activation by α-LA is the result of changes in the amount of NADH and NADHP present in cells, due to the interference of α-LA and/or its analogous derivatives in the oxidation-reduction processes. To verify this hypothesis, use was made of commercial kits (by Valter Occhiena) able to analyze the variations in the cellular amounts of NAD(P)H relative to total NAD(P), in the presence of increasing concentrations of the compounds under analysis (total NAD=NADH+NAD⁺; total NADP=NADPH+NADP⁺).

Methodology
HepG2 cells were seeded in sterile plates (60 mm diameter) at a concentration of 2×10⁵ cells/plate in 4 ml of culture medium (RPMI supplemented with 10% fetal bovine serum (FBS) and enriched with 2 mM of glutamine, 200 U/ml penicillin and 200 U/ml streptomycin);
The plates were incubated at 37° C. at 5% CO₂;
The enantiomers R of the invention, as well as (R)-alpha-lipoic acid, were dissolved in (DMSO) at an initial concentration of 100 mM then diluted directly in the culture medium until the following concentrations were attained: 5, 10, 100 and 500 μM;
After 16-24 hours from seeding, the culture medium was renewed with RPMI+10% FBS containing the specific concentration of the compound under analysis and the corresponding concentration of DMSO as reference;
The culture medium containing the specific concentration of the compound to be assayed was renewed every 24 hours and, after three treatments (corresponding to 72 hours of exposure to the compounds of the invention), the cells were recovered in cold PBS;
Several aliquots of the individual samples were prepared (2×10⁵ cells/aliquot);
Using a spectrophotometric assay, the total NADH/NAD and NADPH/NADP ratios were evaluated for each aliquot, following the instructions accompanying the specific commercial kits (by Valter Occhiena).

Results

FIG. 10 shows the results relating to the amount of NADH (evaluated as total NADH/NAD ratio) obtained after treatment with various compounds ((R)α-LA, compounds of Examples 3a, 5, 11, 7 and 8) given as fold induction relative to the values obtained for the reference control.

The amount of NADH was found to vary with varying concentrations of the compounds employed.

At low concentrations of (R)-α-LA (5 and 10 μM) the amount of NADH was greater than that detected in the control samples, an indication of the fact that under these conditions (R)-α-LA participates as a cofactor in the reduction reactions of NAD⁺ to NADH.

As expected, on increasing the concentration (100 and 500 μm) (R)-α-LA resulted in a decrease in NADH levels. This effect can be explained by hypothesizing that α-LA uses NADH as a reducing agent, to undergo reduction to α-DHLA and by mass effect leads to a reversal of the reaction driven by lipoamide dehydrogenase, thus blocking the oxidative decarboxylation process of pyruvic acid. Under physiological conditions, lipoamide dehydrogenase oxidizes α-DHLA to α-LA using NAD⁺ as the oxidizing agent. In the presence of excess α-LA, the reaction is reversed. The treatment with increasing concentrations of the enantiomers R of Examples 5, 11, 7 and 8 showed a similar NADH pattern to that obtained with (R)-α-LA. In these cases, however, the reduction in NADH resulting from high concentrations of the enantiomers of the invention, was even more extreme.

Exposure to the racemic compound of Example 3a resulted in lower NADH levels than the control, even at a concentration of 5 μM.

In FIG. 11, the results relating to the amount of NADPH after treatment with (R)-alpha-lipoic acid and Examples 3a and 8, are given again as fold induction compared to control. The compounds under analysis also influenced NADPH levels, in a dose dependent manner, highlighting their involvement in the NADP⁺/NADPH oxidation-reduction processes.

From the detailed description and from the aforegiven Examples, the advantages achieved by means of the enantiomers R of the present invention are evident. In particular, said enantiomers R, being less toxic and more pharmacologically active than the corresponding racemic forms, are able to release (R)-alpha-lipoic acid, ensuring a greater bioavailability than that obtainable by direct administration of alpha-lipoic acid itself, or to simulate the pharmacological action of alpha-lipoic acid, while exhibiting a more intense and enduring activity.

1-62. (canceled)

63. An enantiomer R of a compound of formula (I): 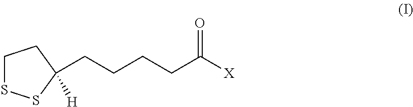
wherein
X is —NH—R₁ , or 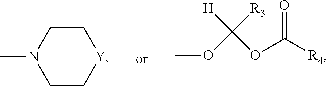
R₁ is —(CH₂)ₙ—R₂,
R₂ is a linear, branched or cyclic C₁-C₆ aliphatic group, —O—(CH₂)ₙ—CH₃, —NH—CO—(CH₂)ₙ—CH₃, a 5- or 6-membered aliphatic or aromatic ring optionally comprising a heteroatom, a 5- or 6-membered aromatic ring substituted by one or two substituents, said substituents being selected from the group consisting of —OH, —O(alkyl C₁-C₃) and —OCO(alkyl C₁-C₃), or 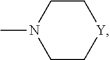
R₃ is H or a C₁-C₃ aliphatic group and R₄ is a linear C₁-C₃ or a branched C₃-C₁₂ aliphatic group, or R₃ is a C₁-C₃ aliphatic group and R₄ is a linear C₁-C₁₂ aliphatic group,
Y is O, CH—(CH₂)ₙ—CH₃ or N(CO)(CH₂)ₙ—CH₃, and
n is an integer from 0 to 6.

64. The enantiomer of claim 63, wherein the compound has formula III, 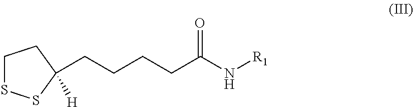
wherein
R₁ is —(CH₂)ₙ—R₂,
R₂ is a linear, branched or cyclic C₁-C₄ aliphatic group, and
n is 0.

65. The enantiomer of claim 64, having formula:

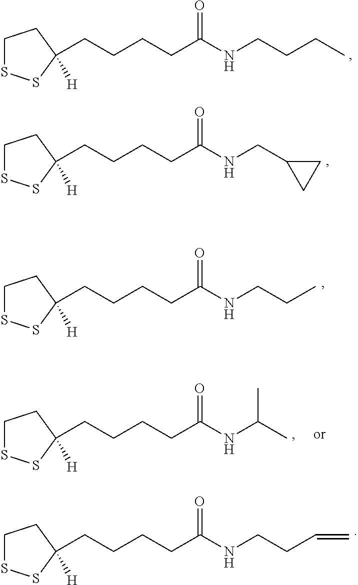

66. The enantiomer of claim 63, having formula III, 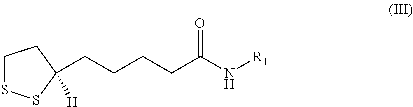
wherein
R₁ is —(CH₂)ₙ—R₂, in which
R₂ is —NH—CO—(CH₂)ₙ—CH₃, a 5- or 6-membered aliphatic ring, a 5-membered aromatic ring, a 5- or 6-membered aromatic ring substituted by one or two substituents, said substituents being selected from the group consisting of: —OH, —O(alkyl C₁-C₃) and —OCO(alkyl C₁-C₃), or 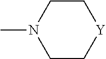
where Y is CH—(CH₂)ₙ—CH₃ or N(CO)(CH₂)ₙ—CH₃, and n is an integer from 0 to 6, or
R₂ is phenyl and n is an integer from 2 to 6, or
R₂ is morpholinyl and n is an integer from 3 to 6, or
R₂ is —O—(CH₂)ₙ—CH₃ and n is an integer from 1 to 6;
or wherein
R₁ is a linear, branched or cyclic C₅-C₁₀ aliphatic group.

67. The enantiomer of claim 66, having formula:

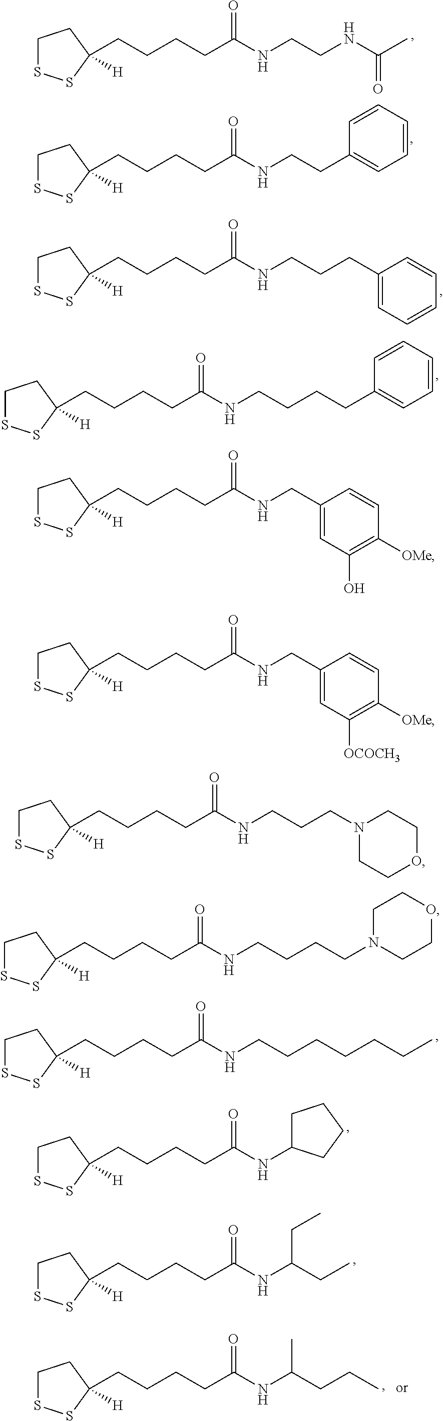

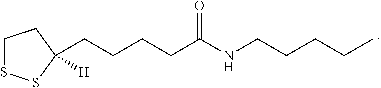

68. The enantiomer of claim 66, wherein R₁ is a linear, branched or cyclic C₇-C₁₀ aliphatic group.

69. The enantiomer of claim 63, having formula II, 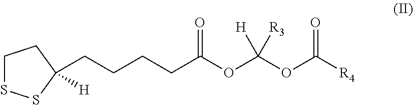
wherein
R₃ is H or a C₁-C₃ aliphatic group and R₄ is a branched C₃-C₁₂ aliphatic group, wherein at least a branch is in alpha-position, or
R₃ is a C₁-C₃ aliphatic group and R₄ is a linear C₁-C₆ aliphatic group.

70. The enantiomer of claim 69 having formula:

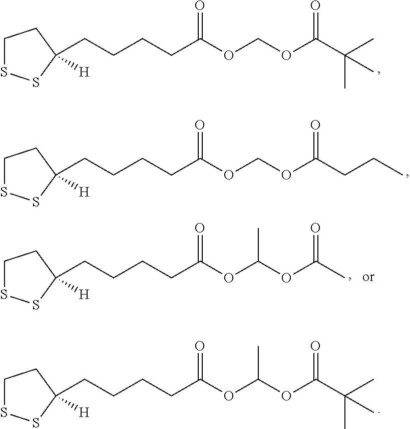

71. The enantiomer of claim 63, having formula IV, 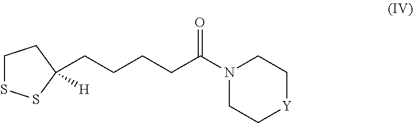
wherein
Y is —CH—(CH₂)ₙ—CH₃ or —N(CO)(CH₂)ₙ—CH₃, and n is an integer from 0 to 3.

72. The enantiomer of claim 71 having formula:

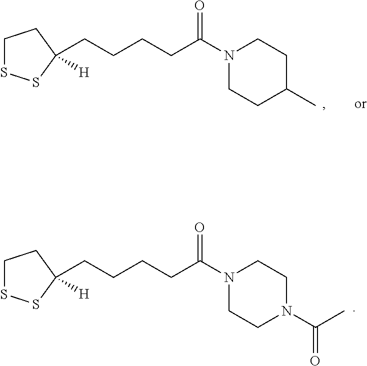

73. A process for preparing the enantiomer of claim 63, the process comprising
reacting (R)-alpha-lipoic acid and a reagent under inert gas atmosphere and room temperature, sheltered from light,
wherein said reagent is selected from the group consisting of NH₂—R₁, 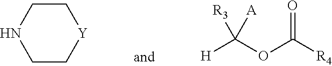
in which
R₁ is —(CH₂)ₙ—R₂,
R₂ is a linear, branched or cyclic C₁-C₆ aliphatic group, —O—(CH₂)ₙ—CH₃, —NH—CO—(CH₂)ₙ—CH₃, a 5- or 6-membered aliphatic or aromatic ring optionally comprising a heteroatom, a 5- or 6-membered aromatic ring substituted by one or two substituents, said substituents being selected from the group consisting of —OH, —O(alkyl C₁-C₃) and —OCO(alkyl C₁-C₃), or 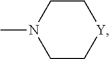
R₃ is H or a C₁-C₃ aliphatic group and R₄ is a linear C₁-C₃ or a branched C₃-C₁₂ aliphatic group, or R₃ is a C₁-C₃ aliphatic group and R₄ is a linear C₁-C₁₂ aliphatic group,
Y is O, CH—(CH₂)ₙ—CH₃ or N(CO)(CH₂)ₙ—CH₃,
A is a halogen; and
n is an integer from 0 to 6.

74. The process of claim 73, wherein the (R)-alpha-lipoic acid and the reagent are in equimolar amounts.

75. A method to treat diabetes, diabetic neuropathy, obesity and pathologies related thereto in an individual, the method comprising
administering to the individual an effective amount of an enantiomer R of the compound of formula I: 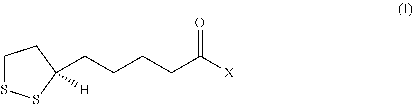
wherein
X is —NH—R₁ or 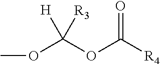
R₁ is —(CH₂)ₙ—R₂, wherein
R₂ is a linear C₁-C₃ aliphatic group and n is an integer from 0 to 2, or
R₂ is a branched or cyclic C₁-C₆ aliphatic group, —O—(CH₂)ₙ—CH₃, —NH—CO—(CH₂)ₙ—CH₃ and n is an integer from 0 to 6, or
R₂ is a 5- or 6-membered aliphatic or aromatic ring optionally comprising a heteroatom, or a 5- or 6-membered aromatic ring substituted by one or two substituents, said substituents being selected from the group consisting of —OH, —O(alkyl C₁-C₃) and —OCO(alkyl C₁-C₃), or 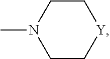
n is an integer from 0 to 6, and
Y is O, CH—(CH₂)ₙ—CH₃ or N(CO)(CH₂)ₙ—CH₃, and
R₃ is H or a C₁-C₃ aliphatic group and R₄ is a branched C₃-C₁₂ aliphatic group, wherein at least a branch is in alpha-position, or R₃ is a C₁-C₃ aliphatic group and R₄ is a linear C₁-C₆ aliphatic group.

76. The method of claim 75, wherein the enantiomer R has formula:

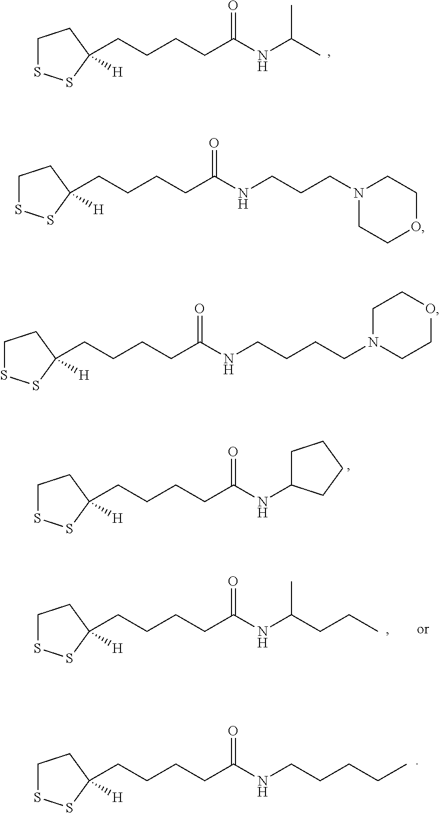

77. The method of claim 75, wherein the enantiomer R has formula I: 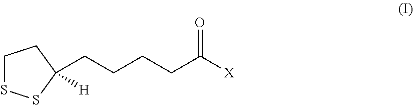
wherein
X is —NH—R₁ or 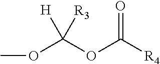
in which
R₁ is —(CH₂)ₙ—R₂, wherein
R₂ is a linear, branched or cyclic C₁-C₃ aliphatic group, and n is 1, or
R₂ is —NH—CO—(CH₂)ₙ—CH₃, —O—(CH₂)ₙ—CH₃, a 5- or 6-membered aliphatic or aromatic ring, a 5- or 6-membered aromatic ring substituted by one or two substituents, said substituents being selected from the group consisting of: —OH, —O(alkyl C₁-C₃) and —OCO(alkyl C₁-C₃), or 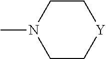
where Y is O, CH—(CH₂)ₙ—CH₃ or N(CO)(CH₂)ₙ—CH₃, and
n is an integer from 1 to 6,
R₃ is H or a C₁-C₃ aliphatic group and R₄ is a branched C₃-C₁₂ aliphatic group, wherein at least a branch is in alpha-position, or
R₃ is a C₁-C₃ aliphatic group and R₄ is a linear C₁-C₆ aliphatic group.

78. The method of claim 77, wherein the enantiomer R has formula:

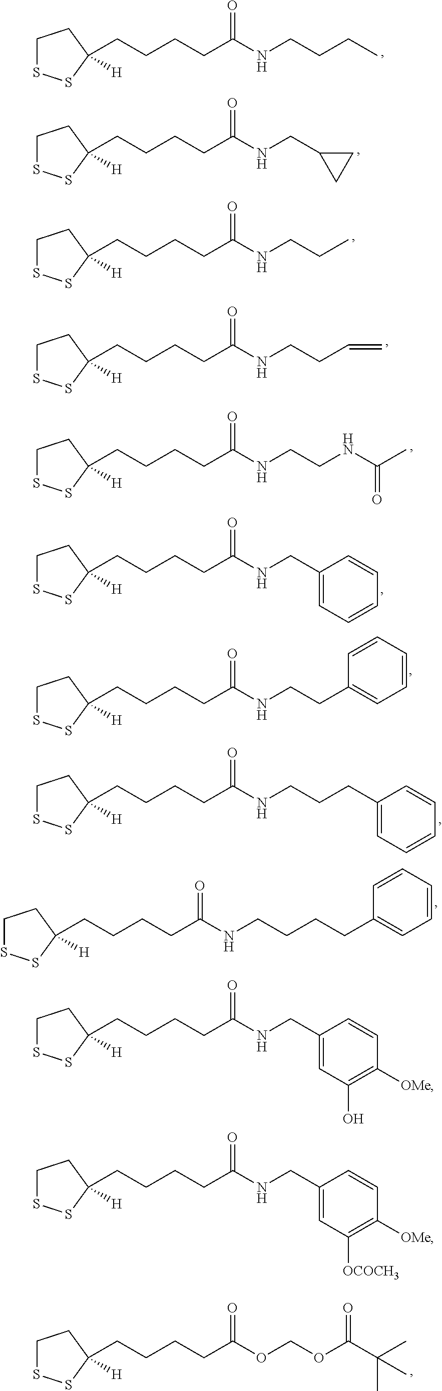

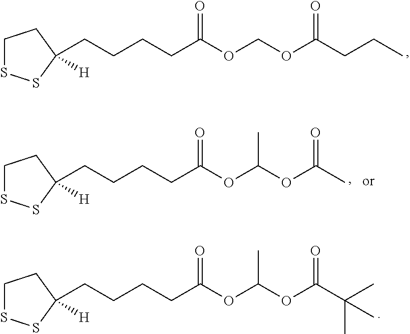

79. Method of treating tumours in an individual, the method comprising
administering to the individual in an amount effective to induce apoptosis of tumour cells, an enantiomer R of a compound of formula I: 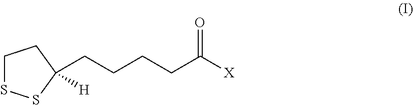
wherein
X is —NH—R₁ or 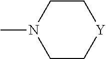
R₁ is a linear C₆-C₁₂ aliphatic group, or is a branched C₅-C₁₂ aliphatic group, wherein at least an ethyl branch is in alpha-position,
Y is O, CH—(CH₂)ₙ—CH₃ or N(CO)(CH₂)ₙ—CH₃, and
n is an integer from 0 to 6.

80. The method of claim 79, wherein the enantiomer R has formula: [chemical structure]
